(19) United States
(12) Patent Application Publication
(10) Pub. No.: US 2020/0078288 A1
(43) Pub. Date: Mar. 12, 2020

(54) BIOBASED, UV-CURABLE NAIL POLISH COMPOSITIONS AND RELATED METHODS

(71) Applicant: EASTERN MICHIGAN UNIVERSITY, Ypsilanti, MI (US)

(72) Inventors: Vijaykumar M. Mannari, Saline, MI (US); Forough Zareanshahraki, Ypsilanti, MI (US)

(21) Appl. No.: 16/563,123

(22) Filed: Sep. 6, 2019

Related U.S. Application Data
(60) Provisional application No. 62/728,193, filed on Sep. 7, 2018.

(51) Int. Cl. A61K 8/87; A61K 8/81; A61K 8/85; A61K 8/36; A61K 8/92; A61Q 3/02; A61K 8/34; A61K 8/40

(52) U.S. Cl. CPC A61K 8/87; A61K 8/8147; A61K 8/85; A61K 8/40; A61K 8/922; A61Q 3/02; A61K 8/345; A61K 8/361

(57) ABSTRACT

The disclosure relates to aqueous and non-aqueous radiation-curable nail coating compositions having a substantial amount of bio-based material in the corresponding polymeric binder. The compositions incorporate a vinyl-functionalized epoxidized bio-based unsaturated compound, which provides substantial bio-based content, vinyl functionality for curing, and soft segment functionality for ease of removal. The aqueous coating compositions generally include (a) a bio-based polymeric binder including a reaction product between a polyurethane pre-polymer and the vinyl-functionalized epoxidized bio-based unsaturated compound, (b) a photoinitiator, and (c) water. The non-aqueous coating compositions generally include (a) a bio-based polymeric binder including the vinyl-functionalized epoxidized bio-based unsaturated compound, a reactive diluent, and a vinyl functional oligomer, and (b) a photoinitiator. Related methods of forming a nail coating are also disclosed.

### CROSS REFERENCE TO RELATED APPLICATION

Priority is claimed to U.S. Provisional Patent Application 62/728,193, filed Sep. 7, 2018, the entire disclosure of which is incorporated herein by reference.

### STATEMENT OF GOVERNMENT INTEREST

None.

### BACKGROUND OF THE DISCLOSURE

### Field of the Disclosure

The disclosure relates to aqueous and non-aqueous radiation-curable nail coating compositions having a substantial amount of bio-based material in the corresponding polymeric binder. The compositions incorporate a vinyl-functionalized epoxidized bio-based unsaturated compound, which provides substantial bio-based content, vinyl functionality for curing, and soft segment functionality for ease of removal.

### Background

Nail polishes are one of the most widely used products in the US cosmetic industry, utilized by 117 million Americans in 2016, which is estimated to reach 122 million by 2020. Gel nail polishes are a specific class of nail polishes, with the ability to be crosslinked under ultraviolet (UV) radiation, and consequently demonstrate improved properties and greater durability compared to the conventional, non-gel nail polishes. Gel nail polishes are usually offered in three layers: basecoat, polish, and clear top-coat. Each layer would be applied after curing the previous layer under radiation from a UV-mercury or UV-LED source.

Valenty et al. U.S. Pat. No. 5,435,994 discloses a radiation curable top-coat composition comprising mainly of nitrocellulose, (meth)acrylate monomers, non-reactive solvents, photoinitiator, inhibitor, etc. to be applied on top of commercial nail enamels.

Goudjil et al. U.S. Pat. No. 5,730,961 discloses a metamorphic radiation curable nail polish consisting of a photochromic compound such as spiroxazine or spiropyran derivatives added to a clear polish comprised a base resin containing nitrocellulose and cellulose acetate butyrate and a photoreactive monomer, that was able to react with UV radiation or sunlight by changing color from dear to any chosen color such us violet, blue, yellow, red, etc. and going back to colorless form upon removed from the ultraviolet source.

Cook et al. U.S. Pat. No. 5,985,951 discloses UV-curable nail coating formulations containing modified cellulose esters with ethylenicaliy unsaturated pendant groups, acrylate monomers or oligomers as copolymerizable reactants, pigments, plasticizers, organic solvent, etc. The coating was formulated to be at least partially soluble in suitable removing solvents.

Vu et al. U.S. Pat. No. 8,901,199 discloses a removable base coat consisting a 3D thermoset lattice dispersed in a network of solvent-dissolvable resin. The thermoset lattice provides durability, toughness and good adhesion, while the solvent-dissolvable resin facilitates removability. For making the 3D lattice, they used copolymers of polymethylmethacrylate and polymethacrylic acid, a solvent-sensitive monomer from polypropyiene/polybutylene glycol (meth)acrylate family, and other acrylate monomers such as urethane (meth)acrylates and cellulose esters were used as the solvent-dissolvable resin. When the polymer was exposed to a solvent, it penetrated to the domains of solvent-sensitive resin, dissolved it and then more easily penetrated to the interior of thermoset matrix.

Kozacheck et al. US20140369944 discloses a storage-stable radiation-curable nail get coating, investigates the effect of different organic and inorganic thixotropic agents on shelf life of pigmented nail gels consisting of urethane acrylate oligomers and (meth)acrytate monomers, and reports a drastic difference in stability of the nail polishes (pigment settlement) with and without thixotropic agents. By changing the rheological properties of the nail gels, the thixotropic agent allows nail gels to be easily applied at lower viscosities due to shear thinning that reduces the amount of required solvent for viscosity adjustment.

Chang et al. US20150190331 discloses a radiation-curable nail lacquer formulation mainly composed of aliphatic/aromatic urethane and polyester acrylate oligomers that contained no irritating reactive (meth)acrylate monomers, possessed good adhesiveness and was easily removable with a wooden or metal stick.

Klang et al. US20170049683 and US20170049684 disclose UV curable nail polish compositions based on aqueous polyurethane dispersions. The prepolymer uses a diisocyanate compound, DMPA, a polyol derived from renewable material, and a compound containing both ethylenic unsaturation and hydroxyl groups. Then after neutralization, the prepolymer was chain extended with a diamine to produce urea linkages, and then was dispersed in water. Final nail compositions were prepared by addition of a photoinitiator, and optionally a leveling agent and a thickener.

Steffier et al. U.S. Pat. No. 8,574,558 discloses UV-curable nail coating formulations based on renewable polyols. The formulations consist mainly of a (meth)acrylate monomer or oligomer prepared from reacting the bio-based poyol with a (meth)acrylate monomer and a co-reactant such as diisocyanate, polyacid, polyester, cyclic lactam, cyclic lactone, epoxy compounds, etc.

### SUMMARY

In an aspect, the disclosure relates to an aqueous radiation-curable nail coating composition comprising: (a) a bio-based polymeric binder comprising a reaction product between (i) a polyurethane pre-polymer having isocyanate end groups (e.g., two opposing terminal isocyanate end group for a linear pre-polymer) and (ii) at least one end-capping compound having at least one hydroxyl group (e.g., for reaction with isocyanate end group to form urethane/carbamate link with prepolymer) and at least one vinyl functional group (e.g., (meth)acrylate group for eventual vinyl polymerization/crosslinking upon exposure to UV radiation); (b) a photoinitiator (e.g., two or more complementary photoinitiators); and (c) water (e.g., as the liquid medium for an aqueous dispersion of the polymeric binder).

The at least one end-capping compound comprises a vinyl-functionalized epoxidized bio-based unsaturated compound selected from the group consisting of unsaturated fatty acids, unsaturated resin acids, esters thereof (e.g., triglyceride ester, alkyl ester such as methyl ester), and combinations thereof. For example, the vinyl-functionalized epoxidized bio-based unsaturated compound can be a (meth)acrylated epoxidized plant or animal oil or fat triglyceride such as soybean oil. Likewise, the vinyl-functionalized epoxidized bio-based unsaturated compound can include a (meth)acrylated epoxidized unsaturated fatty acid or unsaturated resin acid (e.g., rosin mixture of same). The base unsaturated fatty acid or unsaturated resin acid or ester thereof has at least some degree of unsaturation to allow epoxidation of the substrate and subsequent vinyl functionalization of the epoxy groups, for example by esterification with a vinyl carboxylic acid such as (meth)acrylic acid, which is illustrated by AESO in the examples.

The polymeric binder is free of chain extenders and/or has not been prepared with chain extenders (e.g., di- or polyamine, or di- or polyol chain extenders). The polymeric binder generally has only one polyurethane pre-polymer unit per polymeric chain (i.e., as opposed to multiple or several polyurethane pre-polymer units joined by chain extender units different from those included in polyurethane pre-polymer). For example, the polymeric binder suitably has an average number of polyurethane pre-polymer units per polymeric chain in a range from 1 to at most 1.05, 1.1, 1.2, 1.3, or 1.5 (suitably about 1). As a result, the polymeric binder is generally free from urea groups (i.e., no amine-isocyanate reactions from di- or polyamine chain extenders). Notably, the vinyl-functionalized epoxidized bio-based unsaturated compound can have multiple hydroxy functional groups (e.g., as in AESO with about 4-4.5 on average), but it is essentially an end-capping compound that does not extend the polyurethane pre-polymer chain. In particular, the polyurethane pre-polymer and the vinyl-functionalized epoxidized bio-based unsaturated compound are combined in a manner to almost completely arrest chain extension reactions (e.g., by selection of suitable pre-polymer and end capping compound molar ratios). Accordingly the polymeric binder suitably has an average ratio of end capping units to polyurethane pre-polymer units per polymeric chain in a range from 1.5, 1.6, 1.7, 1.8, 1.9, or 1.95 to 2 (suitably about 2). The polymeric binder has a Renewable Raw Material content of at least 40 wt. % (e.g., at least 40 or 50 wt. % and/or up to about 45, 50, 55, 60, 65, or 70 wt. %). The polymeric binder has at least 2 vinyl functional groups resulting from the at least one end-capping compound (e.g., at least 2, 3, 4, 5, or 6 and/or up to 4, 6, 8, 10, or 12 total vinyl end groups, for example discounting possible internal pendant vinyl groups on the polyurethane pre-polymer, to promote for crosslinking during curing).

In an embodiment of the aqueous coating composition, the polyurethane pre-polymer comprises a random copolymer reaction product of: (i) a polyisocyanate (e.g., diisocyanate such as TDI); (ii) a first polyol (e.g., diol) having at least one acid functional group (e.g., carboxylic group such as in DMPA); (iii) a second polyol (e.g., diol) having at least one vinyl functional group (e.g., (meth)acrylate group such as in BPA diacrylate); and (iv) a third polyol (e.g., diol) different from the first and second polyols (e.g., without an acid group and/or without a vinyl functional group; such as a polyester polyol). The different polyols and polyisocyanates provide different attributes of the polyurethane pre-polymer. For example, DMPA (i.e., diol with one carboxylic —COOH group) is used to provide pendent acid functionality to the prepolymer chain, which in turn provides an ionic center (upon neutralizing with a base) for assisting in water dispersibility. The polyol having an acrylate or other vinyl functionality provides a uniform distribution of acrylate or vinyl groups (i.e., rather than only at the pre-polymer chain ends), which may improve properties with fewer stresses and better adhesion in the cured film. In addition, second polyol can be derived from aromatic structures (e.g., bisphenol A) and hence provides a high glass transition temperature hardness to the cured film. In some embodiments, it can be desirable to omit bisphenol A (BPA), whether for safety, regulatory, and/or commercial reasons. Thus, BPA-free biorenewable vinyl esters can also be used. For example, the second polyol can include a vinyl- and hydroxy-functionalized bio-based renewable material such as a plant acid (or resin), plant sugar, sugar alcohol, or a derivative thereof, suitably containing one or more aromatic structures to provide ample hardness and chemical resistance. Examples include rosin-based vinyl esters, such as the product between glycidyl methacrylate (GMA) and fumaric acid-modified rosin, or isosorbide-based vinyl esters, such as the product of the acrylation of isosorbide. Rosin is a plant resin that can serve as the bio-based renewable material. Isosorbide as the bio-based renewable material can be obtained from sorbitol (a sugar alcohol), which can in turn be formed from starch or other source of glucose. Other polyols, such as the third polyol without acid or vinyl functionality can be added for balancing mechanical properties, cost, etc. These polyols additionally can be from bio-based resources, such as a polyol derived from itaconic acid (a bio-based diacid) and diols or polyols to produce a polyester polyol with vinyl functionality pendent to the chains. The total isocyanate/hydroxyl (NCO/OH) equivalent ratio can be selected/controlled for preparing prepolymers of varying molecular weight, varying mechanical properties, and varying end-group content, which in turn affect cured film properties. Typical values for the NCO/OH equivalent (molar) ratio range from 1.25 or 1.35 to 1.6 or 1.75. The polyurethane pre-polymer suitably has a molecular weight in a range from 5000 to 20000 g/mol (e.g., 5000, 8000, 10000, or 12000 g/mol and/or up to 10000, 12000, 16000, or 20000 g/mol). The polymeric binder suitably has a molecular weight in a range from 8000 to 24000 g/mol (e.g., 8000, 10000, 12000, or 14000 g/mol and/or up to 12000, 14000, 18000, or 24000 g/mol). The ratio of polymeric binder molecular weight to polyurethane pre-polymer molecular weight suitably is in range of 1.05 to 1.5 (e.g., at least 1.05, 1.1 or 1.15 and/or up to 1.2, 1.3, 1.4, or 1.5).

In an embodiment of the aqueous coating composition, the at least one end-capping compound further comprises a second (bio-based) end-capping compound having (only) one hydroxyl group and at least two vinyl functional groups (e.g., a polyol that is partially (meth)acrylated or otherwise esterified with vinyl functional groups to have multiple vinyl functionalities and only one remaining hydroxy functionality, thus providing an endcapping group that facilitates crosslinking upon curing). The second end-capping compound can include pentaerythritol triacrylate (PETA) as in the examples. The second end-capping compound can have only one or at least one hydroxyl group (e.g., two or more hydroxyl groups), but the end-capping compound is reacted under conditions/molar ratios such that only one hydroxyl group reacts with the terminal pre-polymer isocyanate group and the second end-capping compound does not perform any substantial degree of chain extension (e.g., as described above). Other second end-capping compounds can include trimethylolpropane diacrylate, dipentaerythritol tetra (or penta) acrylate, or any other type of polymer that contains at least one vinyl group and at least one hydroxyl group, which may or may not be bio-based. Suitable ratios for the first end-capping compounds (e.g., any vinyl-functionalized epoxidized bio-based unsaturated compound(s) such as AESO) to the second end-capping compounds (e.g., PETA) can be about 5:1 to 10:1 on a molar basis (e.g., 6:1 to 9:1 or 7:1 to 8.5:1) or about 70:30 to 40:60 on a weight basis (e.g., 65:35 to 50:50). The first end-capping compound (e.g., AESO) can be about 20-40 wt. % in the total dispersion (e.g., about 60-80 wt. % total solid (polymeric binder) weight).

In an embodiment of the aqueous coating composition, the polymeric binder is present in a range from 40 to 90 wt. % based on the coating composition (e.g., at least 40, 45, 50, 55, 60, or 65 wt. % and/or up to 50, 60, 70, 80, or 90 wt. %). Alternatively or additionally, the water is present in a range from 10 to 60 wt. % based on the coating composition (e.g., at least 10, 15, 20, 25, or 30 wt. % and/or up to 30, 40, 50, or 60 wt. %). Alternatively or additionally, the aqueous coating composition can include total non-volatile matter (NVM) in a range from 20 to 60 wt. % based on the coating composition (e.g., at least 20 or 35 wt. % and/or up to 50 or 60 wt. %).

In an embodiment of the aqueous coating composition, the coating composition further comprises one or more of a thixotropic agent (e.g., HEC, HPMC, other cellulosic polymers), a defoamer (e.g., TEGO FOAMEX 822), an anti-crater and wetting agent (e.g., TEGO TWIN 4200), and a coalescing agent (e.g., diethylene glycol diethyl ether).

In another aspect, the disclosure relates to a non-aqueous radiation-curable nail coating composition comprising: (a) a bio-based polymeric binder comprising: (i) a vinyl-functionalized epoxidized bio-based unsaturated compound selected from the group consisting of unsaturated fatty acids, unsaturated resin acids, esters thereof, and combinations thereof (e.g., same components such as AESO that can be used in the aqueous coating composition), (ii) a reactive diluent having at least one vinyl functional group, and (iii) an (acrylate) oligomer having at least one vinyl functional group; and (b) a photoinitiator (e.g., two or more complementary photoinitiators). In an alternative aspect of the non-aqueous coating composition, the bio-based polymeric binder can comprise: (i) the vinyl-functionalized epoxidized bio-based unsaturated compound, (ii) optionally the reactive diluent, (iii) the oligomer, and (iv) a VOC-exempt organic solvent (e.g., a composition in which the reactive diluent is supplemented with or replaced by the VOC-exempt organic solvent). The polymeric binder has a Renewable Raw Material content of at least 40 wt. % (e.g., at least 40 or 50 wt. % and/or up to about 45, 50, 55, 60, 65, or 70 wt. %). At least one of the vinyl-functionalized epoxidized bio-based unsaturated compound, the reactive diluent, and the oligomer has at least 2 vinyl functional groups (e.g., at least 2, 3, 4, 5, or 6 and/or up to 4, 6, 8, 10, or 12 total vinyl groups to promote for crosslinking during curing). The non-aqueous coating composition generally has a relatively low content of water and/or volatile organic solvents, for example being free or substantially free from water and/or volatile organic solvents. In various embodiments, the non-aqueous coating composition can have not more than 20, 10, 5, 2, 1, 0.5, 0.2, or 0.1 wt. % of either or both components (e.g., and at least 0.01, 0.1, or 1 wt. % either or both components). The coating composition is non-aqueous in the sense that water, if present, is present in relatively small amounts and/or does not form a primary phase (e.g., a continuous phase) of the coating composition. The volatile organic solvents, if present, suitably are VOC-exempt solvents such as acetone and more preferably acetate solvents, such as methyl acetate or t-butyl acetate.

In an embodiment of the non-aqueous coating composition, the reactive diluent can comprise isopropylideneglycerol methacrylate. Reactive diluents can be included more generally in the aqueous and non-aqueous coating compositions. Other mono-, di-, or tri-functional reactive diluents (i.e., based on number of polymerizable ethylenic groups) could also be used in the formulations, as long as they possess low or no skin irritating effects. The reactive diluents suitably can be used in amount of 2 wt. % to 30 wt. % of the coating composition (e.g., at least 2, 4, or 6 wt. % and/or up to 10, 12, 15, 20, or 30 wt. %). In the illustrative examples below, isopropylideneglycerol methacrylate was used in the aqueous and non-aqueous coating compositions as a bio-based, mono-functional monomer to bring flexibility and more bio-content to the system. In the illustrative examples below, trimethylolpropane triacrylate was likewise used in the aqueous coating compositions as a reactive diluent. In some embodiments, the non-aqueous coating composition generally and the reactive diluent more specifically can omit the use of trimethylolpropane triacrylate, which can have a skin-sensitizing effect. In the illustrative examples below, the reactive diluents were used in amounts of about 7-9 wt. % based on the application (e.g., base coat, colored polish coat, top coat). In addition to reactive diluents, VOC-exempt solvents and fast-evaporating solvents such as acetone and acetate solvents (e.g., methyl acetate, t-butyl acetate) can be used, for example to adjust the viscosity. In some embodiments, the coating composition can omit the reactive diluent, with the reactive diluent preferably being replaced with VOC-exempt organic solvents in similar amounts.

In an embodiment of the non-aqueous coating composition, the oligomer comprises at least one of a polyester acrylate oligomer and a polyurethane acrylate oligomer. Suitably, the acrylate oligomer includes a mercapto-modified oligomer (e.g., mercapto-modified polyester acrylate oligomer) to mitigate oxygen inhibition and provide better surface cure. Suitably, multifunctional aliphatic and aromatic urethane acrylate oligomers are used to provide desired acrylate content and also good chemical properties. From the total acrylate oligomer in the polymeric binder, suitably 10-40 wt. % (e.g., at least 10, 15, or 20 wt. % and or up to 20, 25, 30, 35, or 40 wt. %) is a mercapto-modified oligomer, for example with 60-90 wt. % (e.g., at least 60, 70, or 80 wt. % and or up to 80, 85, or 90 wt. %) being (aliphatic and aromatic) urethane acrylates. In the illustrative examples below, the non-aqueous coating composition included about 7-9 wt. % of mercapto-modified polyester acrylate and about 27-31 wt. % of aliphatic/aromatic urethane acrylate, varying between the top coat/polish/base coat formulations. In some cases, the polyurethane acrylate oligomer can be the same or similar to the polyurethane pre-polymer used in the aqueous coating composition, for example only PETA end-capping groups (i.e., no AESO).

In a particular refinement, the polyurethane acrylate oligomer can be synthesized through a non-isocyanate route, for example including (i) a polyurethane reaction product between a poly(cyclic carbonate) monomer and a polyamine monomer, and (ii) an amide reaction product between amine end groups of the polyurethane reaction product and a vinyl-functional carboxylic acid or anhydride thereof. The poly(cyclic carbonate) monomer can include a poly(alkylene oxide) oligomeric backbone, such as based on ethylene glycol and/or propylene glycol, and two, three, or more cyclic carbonate units (e.g., ethylene carbonate group, trimethylene carbonate group). The polyamine monomer can have two, three, or more amine groups (e.g., —NH₂ primary amino groups), for example appended to an alkyl group, a cycloalkyl group, an aromatic group, and combinations thereof. The vinyl-functional carboxylic acid or anhydride can include (meth)acrylic acid or a (meth)acrylic anhydride dimer thereof. By using a non-isocyanate-based oligomer, the corresponding polymeric binder and/or coating composition can be free or substantially free from isocyanate groups (e.g., any residual unreacted isocyanate group functionality from binder or coating composition).

In an embodiment, the oligomer comprises a vinyl ester oligomer comprising an esterification reaction product between (i) a partially esterified epoxidized plant triglyceride, and (ii) a vinyl-functional polycarboxylic acid. The partially esterified epoxidized plant triglyceride can include epoxidized soybean oil or other epoxidized unsaturated triglyceride oil as disclosed herein that is first partially esterified with a carboxylic acid compound, in particular a mono-functional carboxylic acid compound such as rosin acid or benzoic acid. The partially esterified epoxidized plant triglyceride contains at least some remaining epoxide groups. The remaining epoxide groups are then reacted/esterified with a vinyl-functional polycarboxylic acid such as itaconic acid. The vinyl-functional polycarboxylic acid contains at least one vinyl group for reaction with the vinyl groups of the other binder components during radiation curing. The vinyl-functional polycarboxylic acid contains two, three, or more carboxylic acid groups that can link or crosslink different partially esterified epoxidized plant triglycerides (i.e., when the carboxylic acid groups in a single vinyl-functional polycarboxylic acid react with two or more different triglyceride moieties).

In an embodiment of the non-aqueous coating composition, the vinyl-functionalized epoxidized bio-based unsaturated compound is present in a range from about 30 wt. % to about 70 wt. % of the polymeric binder (e.g., at least 30, 35, 40, 45, or 50 wt. % and/or up to 50, 55, 60, 65, or 70 wt. %, such as 30-70 wt. % or 40-60 wt. %). The ranges generally apply to all vinyl-functionalized epoxidized bio-based unsaturated compound species present, when more than one is present. Alternatively or additionally, the (acrylate) oligomer is present in a range from about 20 wt. % to about 70 wt. % of the polymeric binder (e.g., at least 20, 25, 30, 35, 40, 45, or 50 wt. % and/or up to 50, 55, 60, 65, or 70 wt. %, such as 20-70 wt. %, 30-60 wt. %, or 40-60 wt. %). The ranges generally apply to all oligomer species present, when more than one is present. Alternatively or additionally, the reactive diluent is present in a range from about 2 wt. % to about 30 wt. % of the polymeric binder (e.g., at least 2, 4, 6, 10, or 15 wt. % and/or up to 15, 20, 25, or 30 wt. %, such as 2-30 wt. %, 4-20 wt. %, or 6-15 wt. %). While the reactive diluent suitably is present at relatively lower concentrations due to its potential skin irritancy and odor, it is related to the soft segment content (e.g., provided by AESO or otherwise). If the soft segment amount is too high, the desirable hardness can be attained by increasing reactive diluent content to a relatively higher concentration. The ranges generally apply to all reactive diluent species present, when more than one is present. Alternatively or additionally, a weight ratio of the vinyl-functionalized epoxidized bio-based unsaturated compound(s) to the (acrylate) oligomer(s) can be in a range from 0.5 to 2 (e.g., at least 0.5, 0.6, 0.7, 0.8, 0.9, or 1 and/or up to 0.8, 1, 1.2, 1.4, 1.6, 1.8, or 2). Alternatively or additionally, a weight ratio of the vinyl-functionalized epoxidized bio-based unsaturated compound(s) to the reactive diluent(s) can be in a range from 2 to 8 (e.g., at least 2, 2.5, 3, 3.5, or 4 and/or up to 4, 4.5, 5, 6, 7, or 8).

“Bio-based” generally refers to components derived from a plant, animal, microbial, or other biological sources, for example including plant or animal oil or fat triglycerides and derivatives thereof, plant carbohydrates and derivatives thereof, microbial metabolic products such as mono- or poly-hydroxy alcohols, saturated or unsaturated carboxylic acids, and derivatives thereof.

The Renewable Raw Material (RRM) content of the polymeric binder, component of the polymeric binder, component of the curable composition, etc. can be expressed as a relative weight fraction or percent of bio-based material relative to the polymeric binder, component of the polymeric binder, or component of the curable composition as a whole. As described in the examples, the weight percent RRM can be expressed as 100×(weight total RRM components)/(total weight of end product). The weight fraction or percent of bio-based material can be determined based on the weight of bio-based material used during formulation. In general, the RRM values account for bio-based materials having some non-bio-based content. For example for AESO, the base soybean oil is 100% bio-based material. When the soybean oil is subsequently epoxidized and then acrylated with non-bio-based acrylic acid, then some (small) portion of the AESO would be carbon atoms from non-renewable sources, and the corresponding RRM weight of AESO excludes such non-bio-based acrylic content. Alternatively or additionally, the weight fraction or percent of bio-based material can be determined by isotopic assay to determine and compare the ¹⁴C/¹²C ratio of the material with the known ¹⁴C/¹²C ratio for bio-based materials of natural/renewable origin (i.e., 1.0×10⁻¹²). ASTM D 6866 and D 7026 are representative isotopic assays.

Various refinements of the aqueous and non-aqueous coating compositions are possible.

In a refinement, the vinyl-functionalized epoxidized bio-based unsaturated compound comprises a vinyl-functionalized epoxidized triglyceride derived from a plant oil selected from the group consisting of corn oil, canola oil, cottonseed oil, olive oil, safflower oil, palm oil, peanut oil, sesame oil, sunflower oil, soybean oil, and combinations thereof (e.g., a (meth)acrylated ester of an epoxidized derivative of the foregoing oil triglycerides). More generally, the vinyl-functionalized epoxidized triglyceride can be a vinyl-functionalized, epoxidized derivative of a unsaturated fatty acid triglyceride, for example having a combination of unsaturated and saturated fatty acid residues with carbon ranges from 12 to 24 (e.g., at least 12, 14, or 16 and/or up to 16, 18, 20, 22, or 24) and an average degree of unsaturation ranging from 1 to 6 (e.g., at least 1, 2, 3, or 4 and/or up to 3, 3.5, 4, 4.5, 5, or 6). The degree of unsaturation corresponds to the eventual degree of acrylate/vinyl functionality and degree hydroxyl functionality after epoxidation and vinyl-functionalization.

In a refinement, the vinyl-functionalized epoxidized bio-based unsaturated compound comprises acrylated epoxidized-soybean oil (AESO). AESO has approximately 4.0-4.2 acrylate and hydroxyl functionality (typically same number of hydroxyl and acrylate groups present). This number provides suitable acrylate functionality for the product to cure and produce hard film. For example, an analogous composition form acrylated epoxidized palm oil (which has a lower degree of acrylate and hydroxyl functionality), could require longer curing times or additional, higher vinyl functionality components to provide a non-tacky coating after cure. Other plant oils with similar degrees of unsaturation to soybean oil have similarly favorable curing properties. AESO and other acrylated epoxidized plant oils or triglycerides are suitably significant components of both coating compositions because the (i) provide a high bio-based content, (ii) provide soft segment functionality to facilitate removal of the coating from a nail, (iii) have acrylate functionality for curing, (iv) have hyroxyl functionality for polyurethane prepolymer functionalization in the aqueous coating composition, (v) provide good flow properties for ease of application, and (vi) impart good gloss properties to the final cured coatings.

In a refinement, the vinyl-functionalized epoxidized bio-based unsaturated compound comprises a vinyl-functionalized, epoxidized unsaturated fatty acid (e.g., a (meth)acrylated ester of an epoxidized derivative of one or more unsaturated fatty acids). The unsaturated fatty acid has a carbon ranges from 12 to 24 (e.g., at least 12, 14, or 16 and/or up to 16, 18, 20, 22, or 24) and an average degree of unsaturation ranging from 1 to 3 (e.g., at least 1 or 2 and/or up to 2 or 3). The degree of unsaturation corresponds to the eventual degree of acrylate/vinyl functionality and degree hydroxyl functionality after epoxidation and vinyl-functionalization. Suitable precursor unsaturated fatty acids include tall oil fatty acids (TOFA) (primarily oleic acid).

In a refinement, the vinyl-functionalized epoxidized bio-based unsaturated compound comprises a vinyl-functionalized, epoxidized resin acid (e.g., a (meth)acrylated ester of an epoxidized derivative of one or more resin acids). The resin acid is generally unsaturated and can include a multicomponent mixture of resin acids such as in rosin (e.g., as obtained from pine or other plant resins). Illustrative resin acids have three fused 6-carbon rings with 1 or 2 unsaturated bonds (i.e., as sites for epoxidation) and one carboxylic acid group, such as in abietic acid, neoabietic acid, dehydroabietic acid, palustric acid, and/or levopimaric acid (e.g., general formula C₁₉H₂₉COOH) as well as pimaric acid. The degree of unsaturation corresponds to the eventual degree of acrylate/vinyl functionality and degree hydroxyl functionality after epoxidation and vinyl-functionalization.

In a refinement, the photoinitiator comprises a photoinitiator package selected from the group consisting of phosphine oxide, isopropylthioxanthone, copolymerizable amine, and combinations thereof. The photoinitiator package generally includes at least one photoinitiator compound and can include one or more photoinitiator synergists (i.e., a compound that assists the photoinitiator but which does not generally have photoinitiator activity by itself).

In a refinement, the composition further comprises one or more of an inhibitor (e.g., free-radical polymerization inhibitor such as MEHQ) and a cosmetic-grade rheology modifier that does not negatively affect the gloss (e.g., organophilic phyllosilicate or other organic clays). If MEHQ is included, it is preferably present in amount of less than about 10 ppm.

In a refinement, the composition further comprises one or more bio-based components selected from itaconic acid, succinic acid, rosin, polymers thereof, derivatives thereof, and combinations thereof. There are several ways that other bio-based materials can be incorporated. The acid and diacid functionalities can be used to introduce bio-based hydroxy or polyhydroxy functionality into a polymeric binder component. For example, as described above, rosin or other mono-acid could be reacted with a mixture of epoxidized soybean oil (ESO) (or other epoxidized triglyceride or other plant oil) and itaconic acid and/or succinic acid (both bio-based materials) to make bio-based polyester polyols via reaction of epoxy groups with acid (—COOH) groups, which then can be used to make bio-based polyurethane prepolymers as in the aqueous coating composition formulation or can be used as a vinyl ester oligomer in the non-aqueous coating composition formulation. Also, a resulting vinyl ester oligomer prepared from ESO, rosin, and itaconic acid could be radically cured under UV radiation because of the presence of unsaturated double bonds in the structure of itaconic acid, thus contributing the curing capability of the composition. If desired, such oligomer can further be acrylated using the oligomer's hydroxy group to further increase acrylate content (and making the corresponding cured composition harder). This oligomer similarly can be used in the formulation of the non-aqueous, bio-based coating compositions as a binder component. Using similar chemistry, bio-based reactive diluents can be prepared using epoxidized methyl esters of plant oils, and can be incorporated into the binder.

In a refinement, the bio-based polymeric binder is present in a range from about 50 wt. % to about 90 wt. % of the coating composition (e.g., about 55 wt. % to about 85 wt. %, about 60 wt. % to about 80 wt. %, about 65 wt. % to about 75 wt. %, for example about 50, about 55, about 60, about 65, about 70, about 75, about 80, about 85, or about 90 wt. %). The foregoing ranges can apply to the combined amount all polymeric binder components present. Alternatively or additionally, the photoinitiator is present in a range from about 2 wt. % to about 9 wt. % of the coating composition (e.g., about 1-10 wt. %, about 2-9 wt. %, or about 3-7 wt. %, for example about 1, about 2, about 3, about 4, about 5, about 6, about 7, about 8, about 9, about 10 wt. %). The foregoing ranges can apply to the combined amount of all photoinitiator species present, when more than one is present in the composition.

In a refinement, the composition further comprises a pigment. Any conventional pigments are suitable, for example including one or more pigments dispersed in a suitable carrier (e.g., tripropylene glycol diacrylate (TPGDA) monomer carrier or preferably any other lower or non-skin sensitizing type monomer), aqueous pigment dispersions, etc. The pigments can be absent in a clear-coat composition (e.g., as a part of a multi-coat, multi-composition formulation). In a further refinement, the pigment is present in a range from about 1 wt. % to about 10 wt. % of the coating composition (e.g. about 2 wt. % to about 9 wt. %, or about 3 wt. % to about 8 wt. %, for example about 1, about 2, about 3, about 4, about 5, about 6, about 7, about 8, about 9, or about 10 wt. %). The foregoing ranges can apply to each pigment species individually or all pigment species present collectively, when more than one is present in the composition.

In a refinement, the composition has a Renewable Raw Material content of at least 30 wt. % (e.g., at least 30, 40, or 50 wt. % and/or up to about 35, 45, 55, 60, 65, or 70 wt. %). The foregoing ranges apply to the coating composition as a whole, independent of the Renewable Raw Material content of the polymeric binder, which similarly has high Renewable Raw Material content values.

In an aspect, the disclosure relates to a method for coating a nail, the method comprising: (a) applying to a surface of a nail (e.g., fingernail, toenail) the radiation curable coating composition of any of the variously disclosed aspects, embodiments, and refinements (e.g., as an aqueous or non-aqueous composition); (b) subjecting the coated nail to a source of radiation, thereby forming a cured coating on the nail (e.g., via free-radical polymerization and crosslinking of the vinyl functional groups in the polymeric binder); and (c) optionally, repeating steps (a) and (b).

Various refinements of the method for coating a nail are possible.

In a refinement, the source of radiation is one or more of UV-mercury and UV-LED. One or more UV-LED sources (e.g., at differing wavelengths) are particularly suitable as safe UV sources available for use in proximity with human tissue. UV-LED sources are currently used by many salons that use nail gel polishes. UV-mercury lamps (high energy) are suitably used when not in proximity with human tissue (e.g., for an alternative, non-nail substrate), but are used examples to compare the cure efficiency between UV-mercury and UV-LED sources. Within UV-LED sources, there are sources that vary in wavelengths, which can be selected based on the cure response of the formulation. For example, the source can be selected to be compatible with the absorbance spectrum of the particular photoinitiator used in the composition, for example with the radiation source having an emission wavelength that covers or is otherwise at the major or other characteristic absorbance peak for the photoinitiator.

In a refinement, the method further comprises subjecting the coated nail to the source of radiation for a period of time ranging from about 30 seconds to about 60 seconds (e.g., from about 35 s to about 55 s, or about 40 s to about 50 s, for example about 30, about 35, about 40, about 45, about 50, about 55, or about 60 s).

In a refinement, the method further comprises repeating steps (a) and (b) at least one time. For example, the applying and curing/irradiating steps can be repeated at least 1, 2, or 3 times and/or up to 2, 3, or 4 times for a corresponding n+1 total coating layers on the nail (i.e., accounting for the first coating layer prior to step repetition). Different layers can have the same or different polymeric binder and/or same or different other components, such as pigments or absence thereof for colored layers and non-colored/clear layers such as for primers and topcoats.

In a refinement, the method further comprises removing the cured coating from the nail by applying one or more of acetone, methyl acetate, ethyl acetate, and isopropanol alcohol thereto, for example by soaking the nail in a solution of one or more of the foregoing solvents for a period of at least 1, 2, 5, or 10 minutes and/or up to 5, 10, 15, or 20 minutes (e.g., representing an approximate minimum soak time for coating removal). For example, in embodiments, the nail is soaked in a solution for a period of about 5 minutes to about 10 minutes. More generally, the cured coatings easily removable after being soaked by commercial nail polish removers for a few minutes, where commercial nail polish removers usually contain one or more of acetone, ethyl acetate, and isopropanol alcohol.

In a refinement, the method comprising subjecting the coated nail to the source of radiation for a period of 0.5 min to 5 min (e.g., at least 0.5, 0.7, or 1 min and/or up to 1, 1.2, 1.5, 2, 3, 4, or 5 min), wherein the resulting cured coating on the nail is tack-free. Thus, there is no need to wipe the cured coating surface with a solvent (e.g., to eliminate tacky surface portions that might be present in an incompletely cured coating surface). The foregoing irradiation periods can represent a minimum amount of irradiation/curing time, after which the cured coating is tack-free, even though the coating might be further irradiated after formation of the tack-free cured coating. In contrast, conventional gel polishes generally remain tacky after such short periods, even if at least partially cured, and typically would need a wipe-off step with solvent to eliminate the tacky surface portion.

While the disclosed compounds, methods and compositions are susceptible of embodiments in various forms, specific embodiments of the disclosure are illustrated (and will hereafter be described) with the understanding that the disclosure is intended to be illustrative, and is not intended to limit the claims to the specific embodiments described and illustrated herein.

### BRIEF DESCRIPTION OF THE DRAWINGS

FIG. 1 is a schematic of the synthesis of a polyurethane dispersion for the aqueous radiation-curable nail compositions as described herein.

FIG. 2 is a schematic of the components of the bio-based polymeric binder of the non-aqueous radiation-curable nail compositions as described herein.

FIG. 3 is a spider chart showing the overall performance of UV-LED cured non-aqueous and aqueous radiation-curable nail compositions compared to a commercial benchmark.

FIG. 4 is a spider chart showing the overall performance of a 3-layer system of the non-aqueous and aqueous radiation-curable nail compositions compared to a commercial benchmark.

FIG. 5 illustrates a non-isocyanate synthetic route for the formation of urethane acrylate oligomers, for example for use in non-aqueous coating compositions.

FIG. 6 shows the chemical structures of three representative cyclic carbonates (CCs) used in the formation of urethane acrylate oligomers.

FIG. 7 illustrates a synthetic route for the formation of bio-renewable based vinyl ester oligomers, for example for use in non-aqueous coating compositions.

FIG. 8 illustrates a synthetic route for the formation of bio-renewable based vinyl-functional polyols, for example for use in aqueous coating compositions, including (A) a rosin-based vinyl ester oligomer with hydroxyl groups and (B) an isosorbide-based vinyl ester oligomer with hydroxyl groups.

### DETAILED DESCRIPTION

Most gel nail polishes available today are based on petrochemical based resources making them unsustainable. Bio-based materials are excellent renewable resources, with high potential of meeting final-product performance, cost and environmental benefits. In addition to this, bio-based materials can be modified to make them amenable to be cured by advanced UV-LED light that consumes low energy and is safer for human exposure compared to conventional UV-mercury lamps. According to the U.S. Department of Energy (DOE) technology roadmap, 10% of basic chemical building blocks should be derived from plant-based renewable resources by 2020 and this amount should increase to 50% by 2050. Therefore, considering the increasing consumption of nail polishes, there is an unmet need for sustainable nail gel polishes with considerable bio-renewable content.

In an aspect, the disclosure relates to polymers and/or oligomers which have been synthesized from bio-renewable materials such as plant oils (such as soybean oil, corn oil, canola oil), itaconic acid, gum rosin, bio-based succinic acid, to name a few. These bio-based materials and corresponding polymers/oligomers are suitably functionalized with unsaturated functional groups such that they can polymerize and form a crosslinked network when exposed to ultraviolet (UV) radiation, including UV-LED radiation. Using these disclosed polymers oligomers, two representative green UV-LED curable nail gel polish formulations have been developed and are illustrated in the examples: one formulation is a high-solid, non-aqueous, zero-VOC (volatile organic content) composition, and the other formulation is a waterborne, aqueous, polyurethane-based dispersion, both with considerable bio-renewable content. The performance of the two formulations compares favorably with a commercial petro-based benchmark nail polish. Also, both formulations were cured under both UV-mercury and UV-LED radiation sources in order to evaluate their curing efficiency under UV-LED source. The high-solid formulation demonstrated very favorable performance, exceeding that of the benchmark, while waterborne formulation met most of the desirable requirements with some significant technical benefits. The disclosed nail gel polish formulations are greener alternatives to the current products. The disclosed compositions take advantage of environmental and health benefits of UV-LED curing and bio-based oligomers/monomers to provide gel polish compositions with high bio-renewable content that can be cured under UV-LED sources, thus providing low cost and environmentally friendly bio-materials in durable nail-gel applications.

The disclosed compositions have several advantages over other nail polish formulations. (1) The formulations are sustainable compositions, generally containing at least 40 or 50 wt. % of bio-renewable materials (e.g., in the polymeric binder portion of the formulation). (2) The formulations can be cured with UV-LED radiation, which is a safer source of radiation for human health and environment, as compared to UV-mercury sources. (3) Due to oxygen inhibition, many of the commercial UV-LED nail gel formulations remain tacky after being cured under UV-LED light. However, the disclosed formulations rapidly and efficiently cure under UV-LED radiation to obtain completely tack-free surface after generally about 1 minute of radiation using commercially available, low-cost UV-LED systems, because the disclosed formulations are designed to minimize oxygen inhibition. (4) The formulations include both a high-solid, zero-VOC composition, and a waterborne, low-VOC polyurethane-based dispersion. (5) Both formulations showed close cure efficiency under UV-mercury and UV-LED lamps, which means the formulations are properly designed for being cured under UV-LED. (6) Use of irritating (meth)acrylate monomers that could cause adverse allergic reactions, was avoided in formulation of the nail-gels. (7) The zero-VOC, high-solid formulation demonstrated favorable performance, exceeding the petro-based commercial benchmark, and the waterborne formulation met most of the required commercial benchmark properties and demonstrated the ability to be applied as a single-layer nail polish system (e.g., as opposed to a 3-layer base-, color-, and top-coat system for the zero-VOC, high-solid formulation). (8) The waterborne polish could be used in a multilayer system, for example with the waterborne polish as an initial layer on the nail, followed by a high-solid formulation as a topcoat to improve gloss and chemical properties, among others. (9) After application and curing, the nail gel polishes from both the high-solid and waterborne formulations are easily removable after being soaked in commercial nail polish removers (e.g., including one or more of acetone, ethyl acetate, and isopropanol alcohol), for example for 10-15 or 5-10 minutes. (10) The zero-VOC, high-solid formulation has low odor (compared to commercial products), and the waterborne formulation has no odor.

The disclosure relates to aqueous and non-aqueous radiation-curable nail coating compositions having a substantial amount of bio-based material in the corresponding polymeric binder. The compositions incorporate a vinyl-functionalized epoxidized bio-based unsaturated compound, which provides substantial bio-based content, vinyl functionality for curing, and soft segment functionality for ease of removal. The aqueous coating compositions generally include (a) a bio-based polymeric binder including a reaction product between a polyurethane pre-polymer and the vinyl-functionalized epoxidized bio-based unsaturated compound, (b) a photoinitiator, and (c) water. The non-aqueous coating compositions generally include (a) a bio-based polymeric binder including the vinyl-functionalized epoxidized bio-based unsaturated compound, a reactive diluent, and a vinyl functional oligomer, and (b) a photoinitiator. Related methods of forming a nail coating are also disclosed.

### Bio-Based Polymeric Binder

The compositions of the disclosure include a bio-based polymeric binder. As used herein, the term “bio-based” means that the polymeric binder is predominately made up of material(s) derived from living matter (biomass) and either occurs naturally or is synthesized from naturally occurring biomass. Alternatively or additionally, “bio-based” can refer to products made by processes that use biomass. Examples of bio-based materials that can be used to provide the polymeric binder include, for example, plant oils or triglycerides, including but not limited to corn oil, canola oil, cottonseed oil, olive oil, safflower oil, palm oil, peanut oil, sesame oil, sunflower oil, soybean oil, and combinations thereof.

The polymeric binder suitably has a Renewable Raw Material content of at least 40 wt. %. The Renewable Raw Material (RRM) content of a material can be expressed as a relative weight fraction or percent of bio-based content relative to the total weight—inclusive of bio-based and non-bio-based content—of the material. The weight percent RRM can be expressed as 100×(weight total RRM components)/(total weight of end product). The weight fraction or percent of bio-based material can be determined based on the weight of bio-based material used during formulation. In general, the RRM values account for bio-based materials having some non-bio-based content. In embodiments, the polymeric binder has a Renewable Raw Material content of at least about 40 or 50 wt % and/or up to about 45, 50, 55, 60, 65 or 70 wt %, based on the total weight of the polymeric binder, for example, about 40, 42, 45, 47, 50, 52, 55, 57, 60, 62, 65, 67, 68, 69 or 70 wt %.

In embodiments, the composition as a whole has a RRM of at least about 30%. For example, in embodiments, the RRM of the composition (e.g., the aqueous or non-aqueous composition) has an RRM content of at least about 30, 35, 40, 45, 50, 55 or 60%.

The polymeric binder suitably has a molecular weight in a range from 8000 to 24,000 g/mol, for example at least about 8000, 10,000, 12,000, or 14,000 g/mol and/or up to about 12,000, 14,000, 18,000, or 24,000 g/mol, such as 8000, 9000, 10,000, 11,000, 12,000, 13,000, 14,000, 15,000, 16,000, 17,000, 18,000, 19,000, 20,000, 21,000, 22,000, 23,000, or 24,000 g/mol.

The polymeric binder can be present in the composition in an amount ranging from about 50 wt % to about 90 wt %, for example at least about 50, 55, 60, 65, or 70 wt % and/or up to about 65, 70, 75, 80 or 90 wt %, based on the total weight of the coating composition, such as about 50, 55, 60, 65, 70, 75, 80, 85, or 90 wt %.

Vinyl-Functionalized Epoxidized Bio-Based Unsaturated Compound

The polymeric binders described herein include a vinyl-functionalized epoxidized bio-based unsaturated compound. In embodiments, the vinyl-functionalized epoxidized bio-based unsaturated compound can be an unsaturated fatty acid, an unsaturated resin acid, as well as any ester thereof, or any combination thereof.

Suitable unsaturated fatty acids that are vinyl-functionalized and epoxidized include, but are not limited to, triglycerides derived from plant oils such as corn oil, canola oil, cottonseed oil, olive oil, safflower oil, palm oil, peanut oil, sesame oil, sunflower oil, soybean oil, and combinations thereof. Alternatively or additionally, the unsaturated fatty acids that are vinyl-functionalized and epoxidized can include triglycerides derived from fatty acid residues having at least about 12, 14, or 16 and/or up to 16, 18, 20, 22, or 24 carbon atoms, for example about 12, 13, 14, 15, 16, 17, 18, 19, 20, 21, 22, 23, or 24 carbon atoms. The fatty acid residues can be partially saturated and can have an average degree of unsaturation ranging from 1 to 6, for example at least 1, 2, 3, or 4 and/or up to 3, 3.5, 4, 4.5, 5, or 6. In embodiments, the unsaturated fatty acid has an average degree of unsaturation ranging from 1 to 3, for example 1, 1.5, 2, 2.5, or 3. The degree of unsaturation corresponds to the eventual degree of acrylate/vinyl functionality and degree hydroxyl functionality after epoxidation and vinyl-functionalization.

In embodiments, the vinyl-functionalized epoxidized bio-based unsaturated compound includes acrylated epoxidized-soybean oil (AESO). AESO has approximately 4.0-4.2 acrylate and hydroxyl functionality per triglyceride unit. In embodiments, the same number of hydroxyl and acrylate groups are present in the AESO. This number can provide suitable acrylate functionality for the product to cure and produce a hard film. For example, an analogous composition from acrylated epoxidized palm oil which has a lower degree of acrylate and hydroxyl functionality, could require longer curing times or additional, higher vinyl functionality components to provide a non-tacky coating after cure. Other plant oils with similar degrees of unsaturation to soybean oil have similarly favorable curing properties. AESO and other acrylated epoxidized plant oils or triglycerides are suitably significant components of both coating compositions because they (i) provide a high bio-based content, (ii) provide soft segment functionality to facilitate removal of the coating from a nail, (iii) have acrylate functionality for curing, (iv) have hyroxyl functionality for polyurethane prepolymer functionalization in the aqueous coating composition, (v) provide good flow properties for ease of application, and (vi) impart good gloss properties to the final cured coatings.

In embodiments, the vinyl-functionalized epoxidized bio-based unsaturated compound includes a vinyl-functionalized, epoxidized unsaturated fatty acid, for example, a (meth)acrylated ester of an epoxidized derivative of one or more unsaturated fatty acids. The unsaturated fatty acid has a carbon range as described herein and an average degree of unsaturation as described herein, for example a degree of unsaturation ranging from 1 to 3. Suitable precursor unsaturated fatty acids include tall oil fatty acids (TOFA). Crude tall oil can include rosins which include resin acids, such as abietic acid; fatty acids, such as oleic acid, palmitic acid, and linoleic acid; fatty alcohols; unsaponified sterols, and other alkyl hydrocarbon derivatives. After purification and reduction of the tall oil, TOFA can be obtained. In embodiments, the TOFA includes oleic acid.

In embodiments, the vinyl-functionalized epoxidized bio-based unsaturated compound includes a vinyl-functionalized, epoxidized resin acid (e.g., a (meth)acrylated ester of an epoxidized derivative of one or more resin acids). In embodiments, the resin acid is unsaturated and can include a multicomponent mixture of resin acids such as in rosin (e.g., as obtained from pine or other plant resins). Illustrative resin acids have three fused 6-carbon rings with 1 or 2 unsaturated bonds (i.e., as sites for epoxidation) and one carboxylic acid group, such as in abietic acid, neoabietic acid, dehydroabietic acid, palustric acid, and/or levopimaric acid (e.g., general formula C₁₉H₂₉COOH) as well as pimaric acid. The degree of unsaturation corresponds to the eventual degree of acrylate/vinyl functionality and degree hydroxyl functionality after epoxidation and vinyl-functionalization.

Aqueous Radiation-Curable Nail Coating Compositions

In embodiments of the aqueous radiation-curable nail coating composition, the bio-based polymeric binder includes a reaction product between a polyurethane pre-polymer having isocyanate end groups and at least one end-capping compound having at least one hydroxyl group and at least one vinyl functional group.

Polyurethane Pre-Polymer

The polymeric binder generally includes a low molecular weight polyurethane polymer or pre-polymer prepared by a stoichiometric excess of isocyanate (NCO) equivalents over hydroxyl (OH) equivalents. The polymer or pre-polymer can be prepared at the NCO/OH equivalent ratios of 1.05 to 1.5, such as at least 1.05, 1.1, 1.2, or 1.3 and/or up to 1.1, 1.2, 1.3, or 1.5. The polymeric binder can include a single polyurethane pre-polymer unit per polymeric chain (i.e., as opposed to multiple or several polyurethane pre-polymer units joined by chain extender units different from those included in polyurethane pre-polymer). That is, in embodiments, the polymeric binder is free of chain extenders. As used herein, the term “free of chain extenders” means that the polymeric binder suitably contains less than about 5, 4, 3, 2, 1, 0.5, 0.1, or 0.01 wt % chain extenders. Accordingly, the polymeric binder can have an average number of polyurethane pre-polymer units per polymeric chain in a range from 1 to at most 1.05, 1.1, 1.2, 1.3, or 1.5 (suitably about 1). As a result, the polymeric binder is generally free from urea groups (i.e., no amine-isocyanate reactions from di- or polyamine chain extenders). As used herein, the term “generally free from urea groups” means that the polymeric binder suitably contains less than about 10, 5, 4, 3, 2, 1, 0.5, 0.1, or 0.01 wt % of urea groups.

In an embodiment of the aqueous coating composition, the polyurethane pre-polymer comprises a random copolymer reaction product of: (i) a polyisocyanate, (ii) a first polyol (e.g., diol) having at least one acid functional group (e.g., carboxylic group such as in DMPA); (iii) a second polyol (e.g., diol) having at least one vinyl functional group (e.g., (meth)acrylate group such as in BPA diacrylate); and (iv) a third polyol (e.g., diol) different from the first and second polyols (e.g., without an acid group and/or without a vinyl functional group; such as a polyester polyol).

The polyisocyanate can include diisocyanates, triisocyanates, and the like. Examples of suitable polyisocyanates include, but are not limited to, 3,3′-dichloro-4,4′-diisocyanato-1,1′-biphenyl, hexamethylene diisocyanate (HDI), 1,4-phenylene diisocyanate, 1,3-phenylene diisocyanate, m-xylylene diisocyanate, toluene-2,4-diisocyanate (2,4-TDI), tolylene-2,6-diisocyanate (2,6-TDI), poly(hexamethylene diisocyanate), trans-1,4-cyclohexylene diisocyanate, 4-chloro-6-methyl-1,3-phenylene diisocyanate, 1,4-diisocyanatobutane, 1,8-diisocyanatooctane, 1,3-bis(1-isocyanato-1-methylethyl)benzene, 3,3′-dimethyl-4,4′-biphenylene diisocyanate, 1,12-diisocyanatododecane, polyisocyanate, or any combination thereof. In embodiments, the polyisocyanate includes a TDI, such as 2,4-TDI or 2,6-TDI.

Examples of polyols (that can form the basis for any of the first, second, and/or third polyol) include, but are not limited to, poly(ethylene glycol) (PEG), ethylene glycol, diethylene glycol, triethylene glycol, tetraethylene glycol, propylene glycol, dipropylene glycol, tripropylene glycol, 1,3-propanediol, 1,3-butanediol, 1,4-butanediol, neopentyl glycol, 1,6-hexanediol, 1,4-cyclohexanedimethanol, trimethylolpropane, 1,2,6-hexanetriol, triethanolamine, pentaerythritol, glycerol, N,N,N′,N′-tetrakis (2-hydroxypropyl)ethylenediamine, polytetrahydrofuran (PTHF) diol, polytetrahydrofuran (PTHF) triol, polycaprolactone (PCL) diol, polycaprolactone (PCL) triol, polycaprolactone (PCL) polyol, polydimethylsiloxane (PDMS) diol, polydimethylsiloxane (PDMS) triol, polydimethylsiloxane (PDMS) polyol, polyester diol, polyester triol, polyester polyol, polylactide (PLA) diol, polylactide (PLA) triol, polypeptides, polyester, polyether, polyamide, octanediol, fluoroalkane polyol, fluoroalkene polyol, fluoroalkyne polyol, alkane polyol, alkene polyol, alkyne polyol, aromatic polyol, poly(vinyl alcohol), polysaccharide, poly(2-hydroxyethyl methacrylate) (pHEMA), poly(2-hydroxyethyl acrylate), poly(N-Hydroxyethyl acrylamide), poly(N-(Hydroxymethyl)acrylamide), poly(N-tris(hydroxymethyl)methylacrylamide), poly((methyl)acrylate) polyol, poly((methyl)acrylamide) polyol, poly(polytetrahydrofuran carbonate) diol, polycarbonate diol, polycarbonate polyol, or any combination thereof.

Examples of suitable polyols having at least one acid functional group (i.e., the first polyol) include, but are not limited to, dimethylolpropionic acid (DMPA), 2,2-bis(hydroxymethyl)butyric acid, or combinations thereof. Alternatively or additionally, the polyol having at least one acid functional group can include any of the polyols disclosed herein that has been further modified and/or functionalized to include an acid group.

Examples of suitable polyols having at least one vinyl functional group (i.e., the second polyol) include any of the polyols described herein, that has been further modified and/or functionalized with a vinyl group, for example with an acrylate such as methyl acrylate, ethyl acrylate, butyl acrylate, acrylic acid, methylmethacrylate, 2-ethylhexyl acrylate, poly(methyl methacrylate), glycidyl methacrylate (GMA), and the like. The second polyol can further be derived from aromatic compounds, such as bisphenol A (BPA), or from BPA-free vinyl esters, such as rosin-based vinyl esters.

The third polyol can be any polyol, including those described herein, that is different from the first and the second polyol. That is, in embodiments, the third polyol can be any polyol that does not include an acid group. In embodiments, the third polyol can be any polyol that does not include a vinyl functional group. Examples of suitable polyols include any of these described herein, for example, polyester polyol. In embodiments, the third polyol can be derived from bio-based resources, such as a polyol derived from itaconic acid (a bio-based diacid) and diols or polyols to produce a polyester polyol with vinyl functionality pendent to the chains.

The different polyols and polyisocyanates provide different attributes of the polyurethane pre-polymer. For example, the first polyol, such as DMPA, is used to provide pendent acid functionality to the prepolymer chain, which in turn provides an ionic center (upon neutralizing with a base) for assisting in water dispersibility. The second polyol having an acrylate or other vinyl functionality provides a uniform distribution of acrylate or vinyl groups, rather than only at the pre-polymer chain ends, which may improve properties with fewer stresses and better adhesion in the cured film. In addition, the second polyol can be derived from aromatic structures (e.g., bisphenol A) and hence provides a high glass transition temperature hardness to the cured film. Other polyols, such as the third polyol without acid and/or vinyl functionality can be added for balancing mechanical properties, cost, etc.

The total isocyanate/hydroxyl (NCO/OH) equivalent ratio in the polyurethane pre-polymer can be selected/controlled for preparing pre-polymers of varying molecular weight, varying mechanical properties, and varying end-group content, which in turn affect cured film properties. Typical values for the NCO/OH equivalent (molar) ratio in the pre-polymer range from at least about 1.25 or 1.35 and/or up to about 1.6 or 1.75, for example from about 1.25 to about 1.75, about 1.25 to about 1.6, about 1.35 to about 1.6, or about 1.75, such as about 1.25, 1.30, 1.35, 1.40, 1.45, 1.50, 1.55, 1.60, 1.65, 1.70, or 1.75.

The polyurethane pre-polymer suitably has a molecular weight in a range from 5000 to 20,000 g/mol, for example from at least 5000, 8000, 10,000, or 12,000 g/mol and/or up to 10000, 12000, 16000, or 20000 g/mol, such as 5000, 6000, 7000, 8000, 9000, 10,000, 11,000, 12,000, 13,000, 14,000, 15,000, 16,000, 17,000, 18,000, 19,000 or 20,000 g/mol.

The ratio of polymeric binder molecular weight to polyurethane pre-polymer molecular weight suitably is in range of at least about 1.05, 1.1, or 1.15 and/or up to about 1.2, 1.3, 1.4 or 1.5, for example about 1.05, 1.1, 1.15, 1.2, 1.25, 1.3, 1.35, 1.4, 1.45, or 1.5.

End-Capping Compound

As described herein, the polymeric binder is a reaction product of the polyurethane pre-polymer and at least one end capping compound. The at least one end-capping compound includes at least one hydroxyl group and at least one vinyl functional group. In particular, the at least one end-capping compound includes the vinyl-functionalized epoxidized bio-based unsaturated compound described herein.

The polymeric binder includes at least 2 vinyl functional groups resulting from the reaction with the at least one end-capping compound. For example, the polymeric binder can include at least 2, 3, 4, 5, or 6 and/or up to 4, 6, 8, 10, or 12 total vinyl end groups, for example, 2, 3, 4, 5, 6, 7, 8, 9, 10, 11, or 12 vinyl end groups. This number discounts any possible internal pendant vinyl groups that may be included on the polyurethane pre-polymer. The vinyl end groups promote for crosslinking during curing.

In embodiments, the at least one end-capping compound further includes a second end-capping compound having only one hydroxyl group and at least 2 vinyl functional groups. For example, the second end-capping compound can include 1 hydroxyl group and at least 2, 3, 4, 5, 6, 7, 8, 9, 10, 11, or 12 vinyl functional groups.

Non-Aqueous Radiation-Curable Nail Coating Compositions

The non-aqueous radiation-curable nail coating composition also includes a bio-based polymeric binder. In these compositions, the polymeric binder includes the vinyl-functionalized epoxidized bio-based unsaturated compound as described herein. The polymeric binder further includes a reactive diluent having at least one vinyl functional group, and an oligomer having at least one vinyl functional group.

Reactive Diluent

As described herein, the polymeric binder in the non-aqueous composition includes a reactive diluent having at least one vinyl functional group. An example of the reactive diluent is isopropylideneglycerol methacrylate.

The reactive diluent can present in a range from about 2 wt. % to about 30 wt. % of the polymeric binder, for example, at least about 2, 4, 6, 10, or 15 wt. % and/or up to about 15, 20, 25, or 30 wt. %, such as about 2 to about 30 wt. %, about 4 to about 20 wt. %, or about 6 to about 15 wt. %, based on the total weight of the polymeric binder. The reactive diluent suitably is present at relatively lower concentrations due to its potential skin irritancy and odor. The ranges generally apply to all reactive diluent species present, when more than one is present.

The weight ratio of the vinyl-functionalized epoxidized bio-based unsaturated compound(s) to the reactive diluent(s) can be in a range from 2 to about 8, for example, at least about 2, 2.5, 3, 3.5, or 4 and/or up to about 4, 4.5, 5, 6, 7, or 8, such as 2, 2.5, 3, 3.5, 4, 4.5, 5, 5.5, 6, 6.5, 7, 7.5, or 8.

More generally, the reactive diluent can be included in the aqueous and the non-aqueous coating compositions (e.g., as part of the polymeric binder in the nonaqueous composition, or as an additional component in either the aqueous or non-aqueous composition). Other mono-, di-, or tri-functional reactive diluent, based on number of polymerizable ethylenic groups, could also be used in the compositions, as long as they possess low or no skin irritating effects. The reactive diluents suitably can be used in amount of about 2 wt. % to about 30 wt. % of the coating composition, for example, at least about 2, 4, or 6 wt. % and/or up to 10, 12, 15, 20, or 30 wt. %, such as 2, 4, 6, 8, 10, 12, 14, 16, 18, 20, 22, 24, 26, 28, or 30 wt %, based on the total weight of the composition. In addition to reactive diluents, VOC-exempt solvents and fast-evaporating solvents such as acetone can be used.

Oligomer

The polymeric binder of the non-aqueous coating composition further includes an oligomer having at least one vinyl functional group.

In embodiments, the oligomer includes at least one of a polyester acrylate oligomer and a polyurethane acrylate oligomer. Suitably, the acrylate oligomer includes a mercapto-modified oligomer, for example, mercapto-modified polyester acrylate oligomer, to mitigate oxygen inhibition and provide better surface cure. Suitably, multifunctional aliphatic and aromatic urethane acrylate oligomers are used to provide desired acrylate content and also good chemical properties. In embodiments, the oligomer includes a mercapto-modified oligomer and aliphatic and/or aromatic urethane acrylate(s). Based on the total weight of the oligomer in the polymeric binder, about 10 wt % to about 40 wt %, for example, at least about 10, 15, or 20 wt. % and or up to about 20, 25, 30, 35, or 40 wt. %, such as about 10, 15, 20, 25, 30, 35, or 40 wt % can be a mercapto-modified oligomer, while about 60 to about 90 wt. %, for example, at least about 60, 70, or 80 wt. % and or up to about 80, 85, or 90 wt. %, such as 60, 65, 70, 75, 80, 85 or 90 wt % can be the aliphatic and/or aromatic urethane acrylate(s). In embodiments, the polyurethane acrylate oligomer can be the same or similar to the polyurethane pre-polymer used in the aqueous coating composition, for example only PETA end-capping groups (i.e., no AESO).

If the soft segment amount, provided by AESO, for example, in the composition is too high, the desirable hardness can be attained by increasing oligomer content.

In embodiments, the weight ratio of the vinyl-functionalized epoxidized bio-based unsaturated compound(s) to the (acrylate) oligomer(s) can be in a range from about 0.5 to about 2, for example, at least about 0.5, 0.6, 0.7, 0.8, 0.9, or 1 and/or up to about 0.8, 1, 1.2, 1.4, 1.6, 1.8, or 2, such as about 0.5, 0.6, 0.7, 0.8, 0.9, 1.0, 1.1, 1.2, 1.3, 1.4, 1.5, 1.6, 1.7, 1.8, 1.9 or 2.0.

In embodiments of the non-aqueous coating composition, the vinyl-functionalized epoxidized bio-based unsaturated compound can be present in a range of about 30 wt % to about 70 wt % (e.g., at least about 30, 40, 50 wt % and/or up to about 40, 50, 60, or 70 wt %), based on the total weight of the polymeric binder, the oligomer can be present in a range of about 20 wt % to about 70 wt % (e.g., at least about 20, 30, 40, or 50 wt %, and/or up to about 40, 50, 60, or 70 wt %), based on the total weight of the polymeric binder, and the reactive diluent can be present in range of about 2 wt % to about 30 wt % (e.g., at least about 2, 4, 6, 10, or 15 wt % and/or up to about 6, 8, 10, 15, 20 or 30 wt %), based on the total weight of the polymeric binder.

### Photoinitiator

The compositions (i.e., aqueous and non-aqueous compositions) disclosed herein include a photoinitiator or a photoinitiator package. The photoinitiator is present to initiate the curing process of the coating upon radiation with the UV-LED lamp. Examples of suitable photoinitiators include, but are not limited to phosphine oxide, isopropylthioxanthone, copolymerizable amine, or combinations thereof. The photoinitiator package can include at least one photoinitiator compound and can include one or more photoinitiator synergists (i.e., a compound that assists the photoinitiator but which does not generally have photoinitiator activity by itself).

The photoinitiator can be present in an amount ranging from about 2 wt % to about 9 wt %, for example at least about 2, 3, 4, or 5 wt % and/or up to about 6, 7, 8, or 9 wt %, such as about 2, 3, 4, 5, 6, 7, 8, or 9 wt %, based on the total weight of the composition.

In embodiments, the photoinitiator is present in an amount of about 2 wt % to about 9 wt %, based on the total weight of the coating composition, as described herein, and the polymeric binder is present in an amount of about 50 wt % to about 90 wt %, based on the total weight of the coating composition, as described herein.

### Additional Agents

The aqueous radiation-curable nail coating compositions further include water. Water can be included in an amount to make up the balance of the composition. For example, in embodiments, the amount of water in the aqueous composition can range from at least about 10, 15, 20, 25, or 30 wt % and/or up to about 60, 50, 40, 30, or 25 wt %, for example, about 10, 15, 20, 25, 30, 35, 40, 45, 50, 55 or 60 wt. % based on the total weight of the composition.

In embodiments of the aqueous composition, the polymeric binder can be present in a range from about 40 wt % to about 90 wt %, about 45 to about 85 wt %, about 50 wt % to about 75 wt %, and the water is present in a range from about 10 wt % to about 60 wt %, about 20 wt % to about 50 wt %, or about 30 wt % to about 40 wt %, based on the total weight of the coating composition.

In embodiments, the non-aqueous composition is substantially free of water. As used herein, the term “substantially free of water” means that the non-aqueous composition suitably contains less than about 5, 3, 2, 1, 0.5, 0.1 wt % added water, based on the total weight of the composition. It is understood that some ingredients may have residual water content.

In embodiments, the aqueous coating composition further includes one or more of a thixotropic agent, a defoamer, an anti-crater and wetting agent, and a coalescing agent.

The thixotropic agent can be included to assist in imparting sufficient viscosity to the composition under low shear rate conditions to prevent pigment settling, and can show good viscosity reduction upon the applied shear such that good application properties are obtained. The thixotropic agent can include inorganic and/or organic-based materials, as taught in U.S. Patent Application Publication No. 2014/0369944. Examples of thixotropic agents include, but are not limited to, hydroxyethylcellulose (HEC), hydroxypropylmethyl cellulose (HPMC), methylcellulose, ethylcellulose, ethylmethylcellulose, hydroxypropyl cellulose (HPC), hydroxyethyl methyl cellulose, ethyl hydroxyethyl cellulose, carboxymethylcellulose (CMC), or any combination or mixture thereof. In embodiments, the thixotropic agent includes HEC and/or HPMC.

The defoamer can be included to mitigate and/or eliminate the foaming of the composition upon mixing and/or agitation. Examples of suitable defoamers include, but are not limited to, those listed under the TEGO FOAMEX tradename from Evonik Industries, for example TEGO FOAMEX 822.

The anti-crater and wetting agent can be included to help evenly spread and level the aqueous composition across the surface (e.g., of the nail), and to mitigate and/or eliminate the uneven application of the composition to the surface. Examples of suitable anti-crater and wetting agents include, but are not limited to, those listed under the TEGO TWIN tradename from Evonik Industries, for example TEGO TWIN 4200.

The coalescing agent can be included to help bind and optimize the formation of the nail coating upon application. One example of a suitable coalescing agents include, but are not limited to diethylene glycol diethyl ether.

In embodiments, the composition (e.g., the aqueous or the non-aqueous composition) further includes one or more of an inhibitor and/or a rheology modifier. Examples of suitable inhibitors include, but are not limited to free-radical polymerization inhibitors such as MEHQ. If MEHQ is included in the composition, it is preferably present in an amount of less than about 10 ppm. Examples of suitable rheology modifiers include, but are not limited to cosmetic-grade rheology modifiers such as organophilic phyllosilicate or other organic clays. The rheology modifier should be selected such that it does not negatively affect the gloss of the cured composition.

In embodiments, the composition further includes a pigment. The pigment can be any suitable pigment that can impart a particular color to the composition, for example the pigment can include one or more pigments dispersed in a tripropylene glycol diacrylate (TPGDA) monomer carrier or preferably any other lower or non-skin sensitizing type monomer, aqueous pigment dispersions, etc. The pigments can be absent in a clear-coat composition (e.g., as a part of a multi-coat, multi-composition formulation). In a further refinement, the pigment is present in a range from about 1 wt. % to about 10 wt. % of the coating composition, for example, about 2 wt. % to about 9 wt. %, or about 3 wt. % to about 8 wt. %, for example about 1, about 2, about 3, about 4, about 5, about 6, about 7, about 8, about 9, or about 10 wt. %. The foregoing ranges can apply to each pigment species individually or all pigment species present collectively, when more than one is present in the composition.

### Methods of Use

The disclosure further provides methods of coating a nail using the compositions described herein. In particular, the method can include applying to a surface of a nail the radiation curable coating composition described herein (e.g., the aqueous and/or non-aqueous composition), and subjecting the coated nail to a source of radiation, thereby forming a cured coating on the nail. The method can optionally include repeating these steps such that multiple layers of the same or different compositions are applied to the surface of the nail. Each of the aforementioned steps can be repeated any number of times suitable to provide adequate color and or protection to the surface of the nail, for example 0, 1, 2, 3, 4, 5, 6, 7, 9 or 10 times. In embodiments, the method includes repeating the steps at least 1 time.

In embodiments, the source of radiation can be UV-mercury and/or UV-LED.

In embodiments, the method can include subjecting the nail to the source of radiation for a period of time ranging from about 30 seconds to about 60 seconds, for example at least about 30, 45, 40, or 45 seconds and/or up to about 40, 45, 50, 55, or 60 seconds. In embodiments, the method includes subjecting the coated nail to the source of radiation for a period of about 0.5 minutes to about 5 minutes, wherein the resulting cured nail is tack-free. In some embodiments, the coated nail is subjected to the source of radiation for about 0.5 minutes to about 3 minutes.

In embodiments, the method can further include removing the cured coating from the nail. The coating can be removed by applying a removing solution that can include, for example, acetone, methyl acetate, ethyl acetate, isopropanol, or any combination thereof, to the coated nail.

### EXAMPLES

### Materials

Aliphatic polyester polyol (STEPANPOL PC-205P-160, Stepan Co.), Bisphenol A epoxydiacrylate (GENOMER 2252, Rahn USA Corp.), pigment dispersions both aqueous and in reactive diluents (Chromaflo Technologies), GARAMITE 1958 (BYK), CELLOSIZE QP-300 (Dow Chemical), TEGO FOAMEX 822 (Evonik), TEGO TWIN 4200 (Evonik), toluene diisocyante (TDI, Byer), GENOCURE TPO-L (Rahn USA Corp), and isopropyl thioxanthone (ITX, BASF), copolymerizable amine synergist (EBECRYL P115, Allnex), acrylated epoxidized-soybean oil (AESO, EBECRYL 860, Allnex), trimethylolpropane triacrylate (TMPTA, Allnex), and isopropylideneglycerol methacrylate (BISOMER IPGMA, GEO Specialty Chemicals) were used as supplied by their respective manufacturers. Dimethylolpropionic Acid (DMPA), acetone, N-methyl-2-pyrrolidone (NMP), triethylamine (TEA), 4-methoxyphenol (MEHQ), and diethylene glycol diethyl ether were obtained from Sigma Aldrich.

### Example 1—Preparation of an Aqueous Radiation-Curable Nail Composition

### Synthesis of Aqueous Polyurethane Dispersion (PUD)

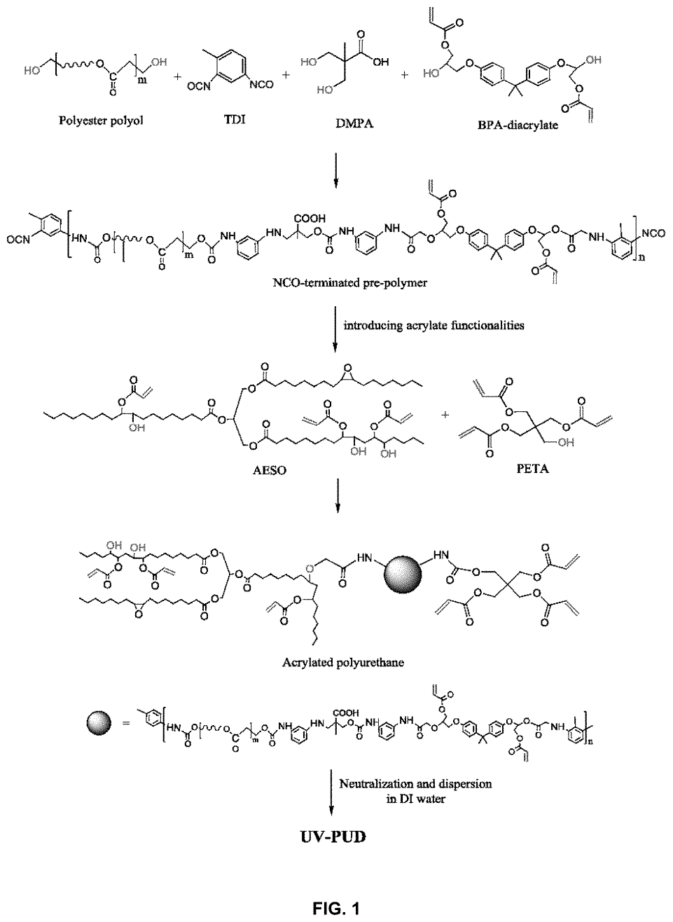

DMPA, aliphatic polyester polyol, Bisphenol A epoxy diacrylate, acetone, and NMP were charged into a three-neck flask equipped with agitator, nitrogen flushing tube, temperature controller, and water-cooled condenser. The contents were heated to 80° C. and held until the solution was homogeneous. TDI was then added drop-wise, and the reaction mixture was reheated to 80° C. and held for one hour. After one hour, the temperature was increased to 90° C. and held to the % NCO target point. The % NCO was determined by the di-n-butylamine back titration method according to ASTM D2572. PETA and AESO were then added to the mixture to introduce acrylate functionality at the chain-ends. The reaction was continued until the desired % NCO (near 0% NCO) was reached. The reaction mixture was then cooled to 40-50° C., and TEA (neutralizing amine) was slowly added and mixed for 5-10 minutes. The neutralized urethane acrylate oligomer was then transferred to the dispersing vessels equipped with a high-speed dispersing agitator. Before dispersing the oligomer in DI water, the oligomer was divided into three proportions that were separately dispersed in DI water. The first one was dispersed without addition of any reactive diluent, to the second one 10% by wt. TMPTA, and to the third one 10 wt % a di-functional acrylate oligomer was added. Agitator speed was increased to 1000-1500 rpm, and de-ionized water was added at a rate sufficient to maintain a vortex. After the complete addition of DI water, agitator speed was reduced to 300-400 rpm, and mixing was continued for an additional 20 minutes. Finally, the polyurethane dispersions obtained were filtered and transferred to plastic containers for storage. A schematic of this process is shown in FIG. 1.

A photoinitiator package including GENOCURE TPO-L and ITX as photoinitiators and EBECRYL P115 as a synergist were added. The structures of these photoinitiators are:

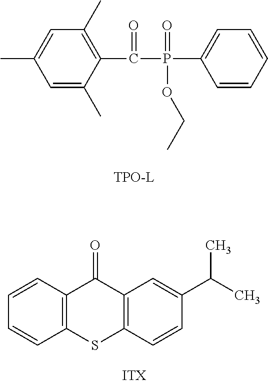 [SMILES: CCOP(=O)(C(=O)c1c(C)cc(C)cc1C)c1ccccc1 ; CC(C)c1ccc2sc3ccccc3c(=O)c2c1]

An aqueous pigment dispersion was selected and added, as was MEHQ as an inhibitor, and Cellosize QP-300 as a thixotropic agent. The composition further included a defoamer, an anti-crater and wetting agent, and a coalescing agent.

The final composition is shown in Table 1, below.

TABLE 1
Composition of Aqueous Radiation-Curable Nail Coating Composition

| | | Weight | Weight |
|---|---|---|---|
| Polish Ingredients | | (gr) | % |
| UV-PUD | Bio-based acrylated | 500 | 80 |
| | polyurethane dispersion | | |
| Amine synergist | Ebecryl P115 | 24.39 | 3.90 |
| Photoinitiator | TPO-L | 15 | 2.40 |
| Package | ITX | 15 | 2.40 |
| Pigments | White pigment dispersion | 30 | 4.80 |
| | Colored pigment dispersion | 9 | 1.44 |
| Additives | Thixotropic agent | 5.3 | 0.85 |
| | Defoamer emulsion | 0.62 | 0.13 |
| | Substrate wetting and anti-crater | 0.6 | 0.09 |
| | additive | | |
| | Coalescing agent | 9.37 | 1.5 |

| | | | |
|---|---|---|---|
| Total = | 625 | g | 100 |

The composition had a Renewable Raw Material content of about 44%, while the PUD independently had a RRM content of 59%.

### Example 2—Preparation of a Non-Aqueous Radiation-Curable Nail Composition

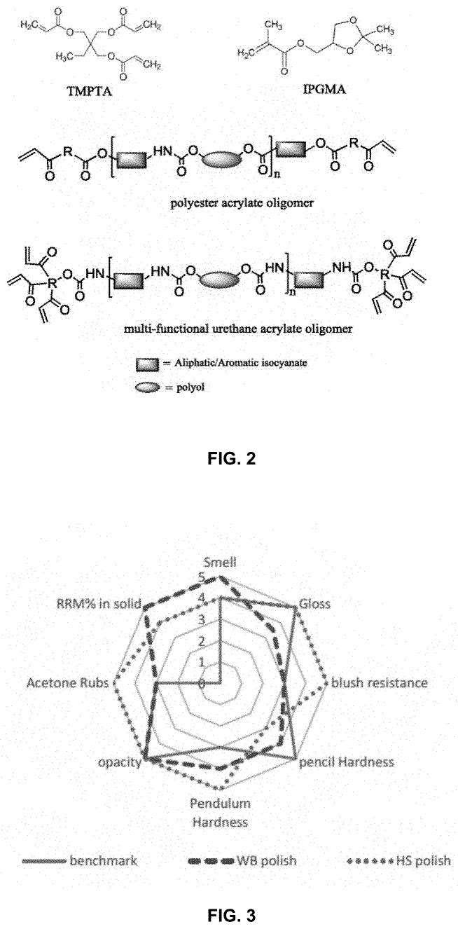

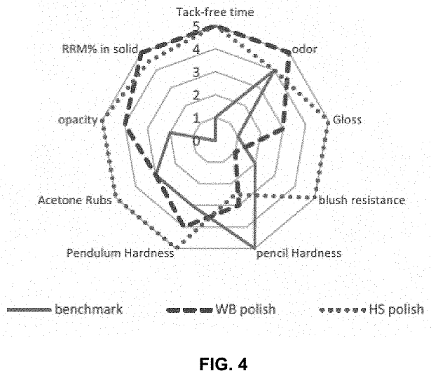

AESO was selected as the bio-renewable based oligomer. A mercapto-modified polyester acrylate oligomer was used to mitigate oxygen inhibition by increasing the cure speed. Multifunctional aliphatic and aromatic urethane acrylate oligomers were used to provide desired acrylate content and also good chemical properties. Chemical structures of the acrylate oligomers and reactive diluents are demonstrated in FIG. 2.

The non-aqueous composition included the same photoinitiator package as described in Example 1.

For pigment, the composition included a tripropylene glycol diacrylate (TPGDA) monomer carrier based pigment dispersion. In other embodiments, the composition can alternatively include a lower or non-skin sensitizing type monomer other than TPGDA as a carrier.

The compositions further included MEHQ as an inhibitor (as described in Example 1), as well as GARAMITE 1958 as a rheology modifier.

The final compositions of the base coat (Table 2; pigment-free), color coat (Table 3), and top coat (Table 4; pigment-free), are provided below.

TABLE 2
Composition of the Non-Aqueous Radiation Curable Composition,
Base coat

| Basecoat | | Weight | |
|---|---|---|---|
| Ingredients | | (gr) | Weight % |
| Binder | AESO | 50 | 45.02 |
| | Acrylate oligomer(s) | 40 | 36.01 |
| | Reactive diluent(s) | 10 | 9 |
| Subtotal = | | 100 | |
| Amine synergist | Ebecryl P115 | 5 | 4.5 |
| Photoinitiator | TPO-L | 3 | 2.7 |
| Package | ITX | 3 | 2.7 |
| Inhibitor | MEHQ | 0.01 | 0.0001 |

| | | | |
|---|---|---|---|
| Total = | 111.06 | g | 100 |

The base coat had a RRM content of about 54%.

TABLE 3
Composition of the Non-Aqueous Radiation Curable Composition,
Color coat

| | | Weight | |
|---|---|---|---|
| Polish Ingredients | | (gr) | Weight % |
| Binder | AESO | 40 | 28.64 |
| | Acrylate oligomer(s) | 40 | 35.80 |
| | Reactive diluent(s) | 10 | 7.16 |
| Subtotal = | | 100 | |
| Amine | Ebecryl P115 | 5 | 3.58 |
| Photoinitiator | TPO-L | 3 | 2.15 |
| Package | ITX | 3 | 2.15 |
| Pigments | White pigment dispersion | 5.35 | 3.83 |
| | Colored pigment dispersion | 1.24 | 0.88 |
| Inhibitor | MEHQ | 0.06 | 0.0001 |
| Additives | Thixotropic agent | 13.1 | 0.75 |

| | | | |
|---|---|---|---|
| Total = | 139.65 | g | 100 |

The color coat had a RRM content of 36%. The Garamite 1958 was dispersed in the binder by 8 wt % prior to adding to the final formulation.

TABLE 4
Composition of the Non-Aqueous Radiation Curable Composition,
Top coat

| Topcoat | | Weight | |
|---|---|---|---|
| Ingredients | | (gr) | Weight % |
| Binder | AESO | 46 | 41.4 |
| | Acrylate oligomer(s) | 34 | 39.6 |
| | Reactive diluent(s) | 10 | 9 |
| Subtotal = | | 100 | |
| Amine | Ebecryl P115 | 5 | 4.5 |
| Photoinitiator | TPO-L | 3 | 2.7 |
| Package | ITX | 3 | 2.7 |
| Inhibitor | MEHQ | 0.011 | 0.0001 |

| | | | |
|---|---|---|---|
| Total = | 111.06 | g | 100 |

The RRM content of the top coat was about 50%.

### Example 3—Curing and Testing of the Radiation-Curable Nail Coating Compositions

### Radiation Curing

SUNUV 48W UV-LED dryer machine with wavelengths in 365 nm and 405 nm, and radiation intensity of 0.691 J/cm² for each 60 seconds of radiation measured by a compact radiometer (UVPS), was used for curing of the gel nail polishes. In addition, in order to evaluate the efficacy of UV-LED curing of the designed formulations, a UV-mercury system (Fusion UV) with an H-bulb with the conveyor belt speed set to 12 feet/min and energy density of ˜0.70 J/cm² per pass was also used.

All the samples were applied at wet film thickness of ˜2 mils on standard 6″×3″ aluminum panels and were cured three times under a 60 second radiation period, or three passes under UV mercury source at 12 feet/min. The aqueous composition was first dried in the oven at 60° C. for 10 min (after 10 min flash-off at room temperature) to remove water before curing under the UV-LED or the UV-mercury lamp. The hardening and eventual full curing of the films were evaluated using a thumb twist procedure, as described in Green et al., “Novel Phosphine Oxide Photoinitiators” (2014). The fully cured films did not leave any observable mark from placing a thumb on the film and twisting.

### Testing & Evaluation

The following tests were performed to evaluate the bio-based gel nail polishes and compare their performance with the petro-based benchmark: Tack-free time, opacity (ASTM D6762), Acetone double-rubs (as described in Vu et al. “Compositions and methods for UV-curable cosmetic nail coatings” (2017)), pendulum hardness test (ASTM D4366), and pencil hardness (ASTM D3363). In addition, blush test (or water resistance) was evaluated by immersion of half coated plates in tap water for 4 hours, and then inspecting them visually after drying. Moreover, the removability of the gel nail polishes was assessed after 10 minutes of immersion in acetone.

Furthermore, the extent of cure for both curing methods (UV-Mercury/UV-LED) was studied by time-based FTIR analysis using a Bruker TENSOR 27 FTIR analyzer. Eight scans were recorded in the range of 400-4000 cm⁻¹. Thin films of nail polishes were applied to prepared KBr pallets, and IR spectroscopy was performed after each pass of curing. To calculate the acrylate double bond conversion, the area of the acrylate band at 810 cm⁻¹ was used. It was normalized using the carbonyl band (1720 cm⁻¹), which is constant throughout polymerization, as a reference peak. A comparison of the ratio of these areas for both the cured and the uncured samples allowed for the the calculation of the extent of acrylate conversion after curing reaction, according to the equation below (Equation 1). Finally, both non-aqueous and aqueous compositions were characterized for gloss at 600 using a micro-TRI Gardco gloss meter.

$$Conversion ( \% ) = 100 \times 1 - ( \frac{{( {A}_{810 {cm}^{- 1}} / {A}_{1720 {cm}^{- 1}} )}_{cured}}{{( {A}_{810 {cm}^{- 1}} / {A}_{1720 {cm}^{- 1}} )}_{uncured}} ) \qquad ( Eq . 1 )$$

Table 5, below, shows the results of evaluation of the commercial benchmark. The base coat, color coat, and top coat of the benchmark were each tacky after 3 passes of radiation at 60 seconds each under UV-LED radiation, and the tackiness problem was not solved after curing for 10 passes. Thus, after three passes, and before the characterization, the very thin tacky layer was wiped off with a paper towel soaked with acetone, as is common in beauty salons. The tackiness was not observed in the UV-mercury curing methods.

TABLE 5
Evaluation of the Commercial Benchmark

| | Acetone | | König Hardness | | Pencil | |
|---|---|---|---|---|---|---|
| | Double Rubs | | (Oscillations) | | Hardness | |
| Method of | UV- | UV- | UV- | UV- | UV- | UV- |
| Curing | mercury | LED | mercury | LED | mercury | LED |
| Base coat | 105 | 100 | 34 | 26 | 3H | HB |
| Color coat | 43 | 20 | 51 | 45 | F | F |
| Top coat | >200 | >200 | 42 | 29 | 6H | 2H |

The results of the evaluation of the non-aqueous radiation-curable coating composition are shown in Table 6, below. In contrast with the benchmark, the non-aqueous coating compositions became tack-free after one minute under UV-LED radiation, which was a considerably superior performance compared to the benchmark. As can be seen in the results, acetone double rubs were in similar range for the layers regardless of the curing method, which shows that curing was performed efficiently under UV-LED radiation. However, the three layers demonstrated higher hardness when cured under UV-mercury radiation, which may have been caused by oxygen inhibition on the surface.

TABLE 6
Evaluation of Non-Aqueous Nail Coating Composition

| | Acetone | | König Hardness | | Pencil | |
|---|---|---|---|---|---|---|
| | Double Rubs | | (Oscillations) | | Hardness | |
| Method of | UV- | UV- | UV- | UV- | UV- | UV- |
| Curing | mercury | LED | mercury | LED | mercury | LED |
| Base coat | 170 | 180 | 126 | 110 | H | 2H |
| Color coat | >200 | >200 | 120 | 114 | F | F |
| Top coat | >200 | >200 | 136 | 120 | 3H | 5H |

The results of the evaluation of the aqueous radiation-curable coating composition are shown in Table 7, below. The aqueous composition, like the non-aqueous composition, was also completely tack-free after the first 60 seconds of curing under UV-LED radiation. As shown by the results, acetone double rub was enhanced considerably by the addition of acrylate monomer/oligomer, inducing more crosslink density. In this hardness, measurements were in a similar range, which shows oxygen inhibition considerably decreased in case of the aqueous composition. This is consistent with other studies that found less or no oxygen inhibition in aqueous systems because of lower solubility of oxygen in water compared to in oil-based formulations.

TABLE 7
Evaluation of Aqueous Nail Coating Composition

| | Acetone | | König Hardness | | Pencil | |
|---|---|---|---|---|---|---|
| | Double Rubs | | (Oscillations) | | Hardness | |
| Method of | UV- | UV- | UV- | | UV- | UV- |
| Curing | mercury | LED | mercury | UV-LED | mercury | LED |
| Polish | 15 | 12 | 86 | 90 | HB | HB |
| Polish + 10 wt % | 45 | 40 | 87 | 94 | F | F |
| TMPTA | | | | | | |
| Non-pigmented | 40 | 38 | 85 | 90 | H | H |
| composition + | | | | | | |
| 10 wt % TMPTA | | | | | | |

All non-aqueous compositions—base coat, color coat, and top coat—passed the blush test regardless of the curing method. However, the aqueous compositions and benchmark compositions failed this test and became hazy after immersion. The addition of 10 wt % TMPTA to the aqueous composition improved the water resistance drastically, which showed that water resistance of the coating improved by increasing the crosslink density.

The UV-LED cured non-aqueous composition was glossy, showing 88.8% gloss at 60°. The aqueous composition was semi-glossy at 71.5% gloss at 60°. The benchmark had the lowest gloss, with a gloss of 20.6% at 60°.

All of the formulations showed good adhesion to the surface and were easily removable from the nail surface after 10 minutes of immersion in acetone.

Based on these results the non-aqueous radiation-curable nail coating composition can be applied even as a single coat and meet the required and cosmetically desired properties for nail gels. In addition, the aqueous radiation-curable nail coating composition offers significant technical benefits, including low odor, high RRMs, and low oxygen inhibition. However, as with the benchmark, this composition needs to be applied with at least about 3 layers in order to demonstrate adequate durability.

### Example 4—Non-Isocyanate Urethane Acrylate Oligomers

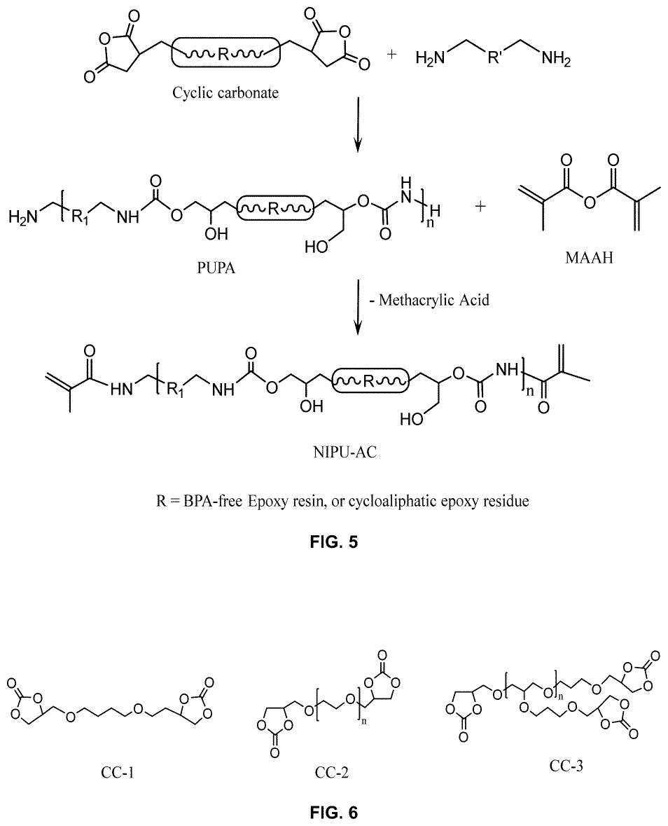

In some embodiments, it may be desirable to avoid the use of isocyanate compounds when forming coating composition components, whether for safety/health reasons or otherwise. Accordingly, this example illustrates an additional form of urethane acrylate oligomers for use in non-aqueous nail gel formulations as disclosed herein, which oligomers can be synthesized through non-isocyanate routes. For example, the urethane acrylate oligomers can be formed using the reaction of cyclic carbonates with excess equivalent ratios of di- or poly-amines to achieve polyurethane polyamines (PUPAs), followed by methacrylation of the amine groups with methacrylic anhydride (MAAH) as illustrated in FIG. 5.

Synthesis of and Characterization of Multifunctional Cyclic Carbonates (MF-CCs):

In this example, multifunctional cyclic carbonates were synthesized by carbonation of epoxy compounds. The catalyst (MePh.I by 2 to 5 mol. % of the epoxy) was dissolved in a solution of epoxide in an alcoholic solvent. Carbon dioxide was purged into the flask at 1 atm pressure, and the reaction mixture was stirred at 70° C. When the reaction was complete, as indicated by complete consumption of epoxide groups, the mixture was cooled to room temperature, and the solvent and catalyst were removed using hot water/ethyl acetate in a separatory funnel. The ethyl acetate phase containing cyclic carbonates was dried over anhydrous sodium sulfate, and the product was isolated by vacuum distillation of the solvent. The progress of the reaction was tracked using oxirane oxygen content (OOC %) titration according to ASTM D1652 standard and also by Fourier Transform Infrared (FTIR) analysis. FIG. 6 shows the chemical structures of the three different cyclic carbonates (CC1-CC3) synthesized from the respective epoxy compounds and used in the formation of urethane acrylate oligomers.

Synthesis of and Characterization of Non-Isocyanate Polyurethane Polyamines (NIPU-PAs):

In order to derive amine-functional non-isocyanate polyurethanes (NIPUs), cyclic carbonates CC1-CC3 were reacted with diamines IPDA (or isophorone diamine) via a step-growth polymerization reaction using an excess equivalent ratio of amine/cyclic carbonate. This reaction was carried out in a three-neck flask equipped with a mechanical stirrer, an inlet for nitrogen, a temperature controller probe, and a water condenser setup. Cyclic carbonate and the calculated amount of amine were dissolved in toluene and added to the reaction flask. The equivalent weights of cyclic carbonates were calculated from that of the corresponding epoxy compounds, which was calculated by the titrimetric method. The reaction temperature was then raised to 90° C. and mechanically stirred during the entire course of the reaction. The reaction conversion was tracked by amine-value titration according to ASTM D2074 standard. The obtained non-isocyanate polyurethane polyamines (NIPU-PAs) were characterized by FTIR and by determination of their Amine Hydrogen Equivalent Weight (AHEW). Table 8 summarizes the characteristics of the developed NIPU-PAs.

TABLE 8
Characteristics of Synthesized NIPU-PAs

| | | | Amine/CC | Amine |
|---|---|---|---|---|
| | Type | Type of | equivalent | equivalent |
| NIPU-PA Naming | of CC | Amine | ratio | weight |
| NIPU-PA (CC1-IPDA-1.7) | CC1 | IPDA | 1.7 | 406 |
| NIPU-PA (CC2-IPDA-1.7) | CC2 | IPDA | 1.7 | 944 |
| NIPU-PA (CC3-IPDA-1.7) | CC3 | IPDA | 1.7 | 1448 |

Methacrylation of NIPU-PAs with MAAH:

In order to synthesize the non-isocyanate polyurethane acrylates (NIPU-ACs), NIPU-PA, toluene, and BHT (0.25 wt % of total solid) as an inhibitor were charged into a three-neck flask equipped with a temperature controller, a condenser, and a nitrogen inlet. Then, MAAH (1:1 equivalent ratio to amine) was added drop-wise, while the flask was kept in an ice bath to control the temperature rise due to the highly exothermic reaction. After the complete addition, the temperature was raised to 60° C. The progress in the reaction was monitored by amine value titration and FTIR spectroscopy to trace the changes in the anhydride peak (1780-1790 cm⁻¹). The reaction was continued until the amine value reached close to zero and the anhydride peak disappeared. After the completion of the reaction, methacacrylic acid, which was produced as a by-product, and toluene were removed at reduced pressures using a vacuum pump. Acetate solvents such as methyl acetate or butyl acetate were used to adjust the viscosity of oligomers, if needed. Table 9 summarizes the characteristics of the developed NIPU-ACs. In the experiments shown in Table 9, equivalent ratios of amine/CC could be changed between 1.1 and 1.9 in order to get NIPU-ACs with different acrylate equivalent weights.

TABLE 9
Characteristics of the synthesized NIPU-ACs

| Naming of | | acrylate equivalent |
|---|---|---|
| NIPU-ACs | Type of PUPA | weight |
| NIPU-AC-1 | NIPU-PA (CC1-IPDA-1.7) | 474 |
| NIPU-AC-2 | NIPU-PA (CC2-IPDA-1.7) | 1012 |
| NIPU-AC-3 | NIPU-PA (CC3-IPDA-1.7) | 1516 |

### Example 5—Bio-Renewable-Based Vinyl Ester Oligomers

In some embodiments, polyester acrylate oligomers used in the formulation of non-aqueous nail gels could also be selected from bio-renewable vinyl ester oligomers. Such oligomers can be formed through the partial esterification of some (but not all) epoxy groups in an epoxidized soybean oil (ESO) structure with different acids (such as rosin acid, succinic acid, benzoic acid, and adipic acid), followed by introduction of vinyl groups via reaction of the remaining epoxy functionalities with a vinyl-functional polycarboxylic acid (e.g., having 2, 3 or more carboxylic acid groups and at least 1 vinyl group) such as itaconic acid. Instead of ESO, other modified epoxidized plant oils, triglycerides, polysaccharides or sugars, or sugar alcohols could be used, for example epoxidized sorbitol.

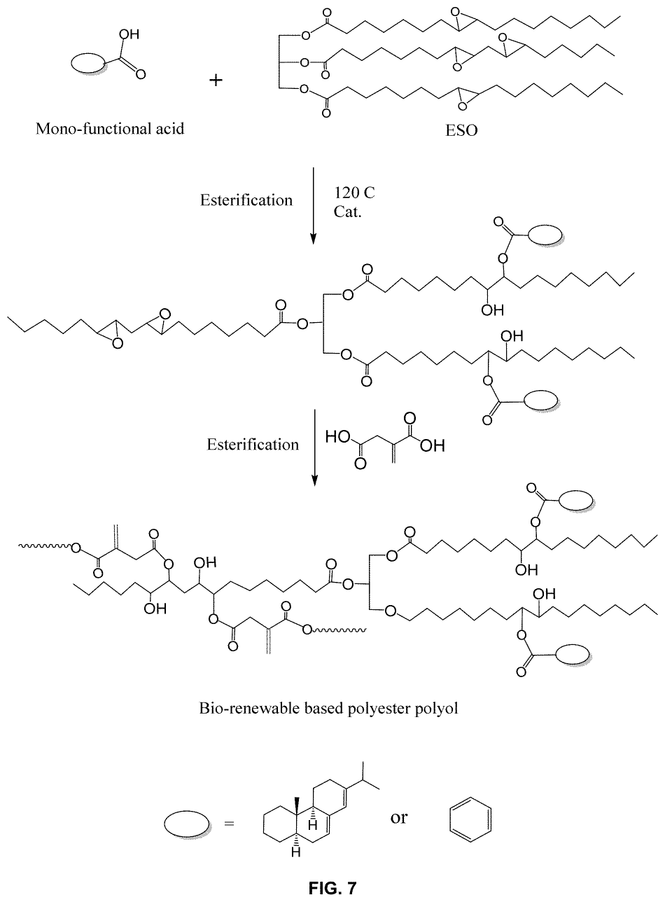

As illustrated in FIG. 7, bio renewable-based vinyl ester oligomers in this example were prepared via a two-step procedure in order to prevent gelation. If di- or multifunctional acids are added to ESO in one step, there is a high chance of gelation due to high average functionality. Therefore, in the approach illustrated in this example, the average degree of epoxide functionality in ESO was first reduced by a desired extent via first reacting the ESO with a mono-functional acid compound, such as rosin or benzoic acid. As illustrated in FIG. 7 (intermediate product), such partial reaction with a monoacid converts some of the epoxide groups to pendant ester groups and hydroxyl groups, while some other epoxide groups remain.

In the first step illustrated in FIG. 7, one equivalent of ESO was charged into a three-neck flask equipped with a nitrogen inlet, thermometer, and condenser. Then, NACURE XC-9206, the esterification catalyst, was added. The reaction temperature was raised to 120° C., and gum rosin or benzoic acid was added in a specific equivalent ratio to ESO. The reaction progress was tracked by acid value and OOC titrations according to ASTM D874 and ASTM D1652, respectively. The reaction was continued until the acid value reached close to zero. In the second step illustrated in FIG. 7, a corresponding amount of itaconic acid was added for chain extension via reaction with the residual epoxy groups, based on the final OOC number. The reaction was continued until reaching an OOC value near zero. Table 10 presents some example compositions of the vinyl ester oligomers formed in this example which can be used as the polyester acrylate oligomers of the non-aqueous coating compositions disclosed herein.

TABLE 10
Composition of Vinyl Ester Oligomers

| Vinyl | | | | |
|---|---|---|---|---|
| ester oligomer | Equivalent of each component | | | |
| naming | Rosin | ESO | Benzoic acid (BA) | Itaconic Acid (IA) |
| VES-1 | 0.35 | 1 | — | 0.25 |
| VES-2 | 0 | 1 | 0.35 | 0.23 |
| VES-3 | 0.20 | 1 | 0.15 | 0.23 |

### Example 6—Bio-Renewable-Based Vinyl-Functional Polyols

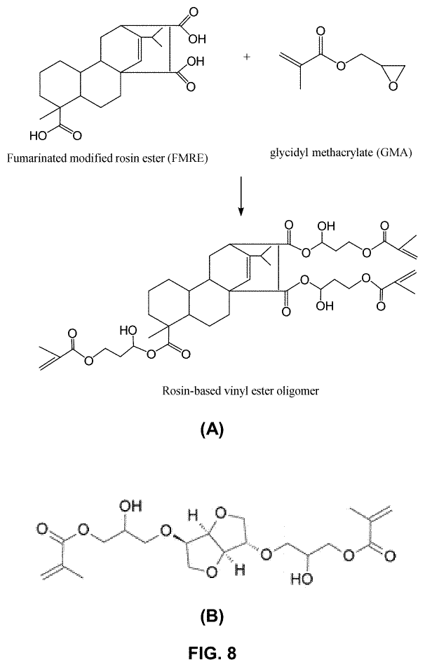

The aqueous coating compositions according to the disclosure include a polyurethane pre-polymer which can be a random copolymer reaction product of a polyisocyanate with first, second, and third polyols, where the second polyol has at least one vinyl functional group. This example illustrates bio-renewable-based vinyl-functional polyols that can be used as the second polyol in a polyurethane pre-polymer and corresponding aqueous coating composition. One possible route to synthesize bio-renewable based vinyl ester polyols is through the reaction between glycidyl methacrylate (GMA) and fumaric acid-modified rosin, as shown in FIG. 8 (panel A). FIG. 8 (panel B) also illustrates an isosorbide-based vinyl ester polyol (isosorbide diglycidyl methacrylate or ISDGMA), which can be formed by first epoxidizing isosorbide and then by second esterification with methacrylic acid.

Because other modifications and changes varied to fit particular operating requirements and environments will be apparent to those skilled in the art, the disclosure is not considered limited to the example chosen for purposes of illustration, and covers all changes and modifications which do not constitute departures from the true spirit and scope of this disclosure.

Accordingly, the foregoing description is given for clearness of understanding only, and no unnecessary limitations should be understood therefrom, as modifications within the scope of the disclosure may be apparent to those having ordinary skill in the art.

All patents, patent applications, government publications, government regulations, and literature references cited in this specification are hereby incorporated herein by reference in their entirety. In case of conflict, the present description, including definitions, will control.

Throughout the specification, where the compounds, compositions, methods, and processes are described as including components, steps, or materials, it is contemplated that the compositions, processes, or apparatus can also comprise, consist essentially of, or consist of, any combination of the recited components or materials, unless described otherwise. Component concentrations can be expressed in terms of weight concentrations, unless specifically indicated otherwise. Combinations of components are contemplated to include homogeneous and/or heterogeneous mixtures, as would be understood by a person of ordinary skill in the art in view of the foregoing disclosure.

What is claimed is:

1. An aqueous radiation-curable nail coating composition comprising:
(a) a bio-based polymeric binder comprising a reaction product between (i) a polyurethane pre-polymer having isocyanate end groups and (ii) at least one end-capping compound having at least one hydroxyl group and at least one vinyl functional group, wherein:
the at least one end-capping compound comprises a vinyl-functionalized epoxidized bio-based unsaturated compound selected from the group consisting of unsaturated fatty acids, unsaturated resin acids, esters thereof, and combinations thereof,
the polymeric binder is free of chain extenders,
the polymeric binder has a Renewable Raw Material content of at least 40 wt. %, and
the polymeric binder has at least 2 vinyl functional groups resulting from the at least one end-capping compound;
(b) a photoinitiator; and
(c) water.

2. The coating composition of claim 1, wherein the polyurethane pre-polymer comprises a random copolymer reaction product of:
(i) a polyisocyanate;
(ii) a first polyol having at least one acid functional group;
(iii) a second polyol having at least one vinyl functional group; and
(iv) a third polyol different from the first and second polyols.

3. The coating composition of claim 2, wherein the second polyol comprises a vinyl- and hydroxy-functionalized bio-based renewable material selected from the group consisting of plant acids, plant sugars, sugar alcohols, and derivatives thereof.

4. The coating composition of claim 1, wherein the at least one end-capping compound further comprises a second end-capping compound having (only) one hydroxyl group and at least two vinyl functional groups.

5. The coating composition of claim 1, wherein:
the polymeric binder is present in a range from 40 to 90 wt. % based on the coating composition; and
the water is present in a range from 10 to 60 wt. % based on the coating composition.

6. The coating composition of claim 1, further comprising one or more of a thixotropic agent, a defoamer, an anti-crater and wetting agent, and a coalescing agent.

7. A non-aqueous radiation-curable nail coating composition comprising:
(a) a bio-based polymeric binder comprising: (i) a vinyl-functionalized epoxidized bio-based unsaturated compound selected from the group consisting of unsaturated fatty acids, unsaturated resin acids, esters thereof, and combinations thereof, (ii) a reactive diluent having at least one vinyl functional group, and (iii) an oligomer having at least one vinyl functional group, wherein
the polymeric binder has a Renewable Raw Material content of at least 40 wt. %, and
at least one of the vinyl-functionalized epoxidized bio-based unsaturated compound, the reactive diluent, and the oligomer has at least 2 vinyl functional groups; and
(b) a photoinitiator.

8. The coating composition of claim 7, wherein the reactive diluent comprises isopropylideneglycerol methacrylate.

9. The coating composition of claim 7, wherein the oligomer comprises at least one of a polyester acrylate oligomer and a polyurethane acrylate oligomer.

10. The coating composition of claim 9, wherein:
the oligomer comprises the polyurethane acrylate oligomer; and
the polyurethane acrylate oligomer is a non-isocyanate oligomer comprising (i) a polyurethane reaction product between a poly(cyclic carbonate) monomer and a polyamine monomer, and (ii) an amide reaction product between amine end groups of the polyurethane reaction product and a vinyl-functional carboxylic acid or anhydride thereof.

11. coating composition of claim 7, wherein the oligomer comprises a vinyl ester oligomer comprising an esterification reaction product between (i) a partially esterified epoxidized plant triglyceride, and (ii) a vinyl-functional polycarboxylic acid.

12. The coating composition of claim 7, wherein:
the vinyl-functionalized epoxidized triglyceride is present in a range from about 30 wt. % to about 70 wt. % of the polymeric binder;
the oligomer is present in a range from about 20 wt. % to about 70 wt. % of the polymeric binder; and
the reactive diluent is present in a range from about 2 wt. % to about 30 wt. % of the polymeric binder.

13. The coating composition of claim 1, wherein the vinyl-functionalized epoxidized bio-based unsaturated compound comprises a vinyl-functionalized epoxidized triglyceride derived from a plant oil selected from the group consisting of corn oil, canola oil, cottonseed oil, olive oil, safflower oil, palm oil, peanut oil, sesame oil, sunflower oil, soybean oil, and combinations thereof.

14. The coating composition of claim 1, wherein the vinyl-functionalized epoxidized bio-based unsaturated compound comprises acrylated epoxidized-soybean oil.

15. The coating composition of claim 1, wherein the vinyl-functionalized epoxidized bio-based unsaturated compound comprises a vinyl-functionalized, epoxidized unsaturated fatty acid.

16. The coating composition of claim 1, wherein the vinyl-functionalized epoxidized bio-based unsaturated compound comprises a resin acid.

17. The coating composition of claim 1, wherein the photoinitiator comprises a photoinitiator package selected from the group consisting of phosphine oxide, isopropylthioxanthone, copolymerizable amine, and combinations thereof.

18. The coating composition of claim 1, further comprising one or more of an inhibitor and a rheology modifier.

19. The coating composition of claim 1, further comprising one or more bio-based components selected from itaconic acid, succinic acid, rosin, polymers thereof, derivatives thereof, and combinations thereof.

20. The coating composition of claim 1, wherein:
the bio-based polymeric binder is present in a range from 50 wt. % to 90 wt. % of the coating composition; and
the photoinitiator is present in a range from 2 wt. % to 9 wt. % of the coating composition.

21. The coating composition of claim 1, further comprising a pigment.

22. The coating composition of claim 21, wherein the pigment is present in a range from 1 wt. % to 10 wt. % of the coating composition.

23. The coating composition of claim 1, wherein the composition has a Renewable Raw Material content of at least 30 wt. %.

24. A method for coating a nail, the method comprising:
(a) applying to a surface of the nail the radiation curable coating composition of claim 1;
(b) subjecting the coated nail to a source of radiation, thereby forming a cured coating on the nail; and
(c) optionally, repeating steps (a) and (b).

25. The method of claim 24, wherein the source of radiation is one or more of UV-mercury and UV-LED.

26. The method of claim 24, comprising subjecting the coated nail to the source of radiation for a period of time ranging from about 30 seconds to about 60 seconds.

27. The method of claim 24, comprising repeating steps (a) and (b) at least one time.

28. The method of claim 24, further comprising removing the cured coating from the nail by applying one or more of acetone, methyl acetate, ethyl acetate, and isopropanol alcohol thereto.

29. The method of claim 24, comprising subjecting the coated nail to the source of radiation for a period of 0.5 min to 5 min, wherein the resulting cured coating on the nail is tack-free and the cured coating is not further wiped with solvents.

30. The method of claim 29, wherein the coated nail is subjected to the source of radiation for a period of 0.5 min to 3 min.

31. The coating composition of claim 7, wherein the vinyl-functionalized epoxidized bio-based unsaturated compound comprises a vinyl-functionalized epoxidized triglyceride derived from a plant oil selected from the group consisting of corn oil, canola oil, cottonseed oil, olive oil, safflower oil, palm oil, peanut oil, sesame oil, sunflower oil, soybean oil, and combinations thereof.

32. The coating composition of claim 7, wherein the vinyl-functionalized epoxidized bio-based unsaturated compound comprises acrylated epoxidized-soybean oil.

33. A method for coating a nail, the method comprising:
(a) applying to a surface of the nail the radiation curable coating composition of claim 7;
(b) subjecting the coated nail to a source of radiation, thereby forming a cured coating on the nail; and
(c) optionally, repeating steps (a) and (b).
